(19) United States
(12) Patent Application Publication
(10) Pub. No.: US 2005/0074426 A1
(43) Pub. Date: Apr. 7, 2005

(54) Fusions of cytokines and tumor targeting proteins

(76) Inventors: Angelo Corti, Milan (IT); Flavio Curnis, Milan (IT)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/492,144

(22) PCT Filed: Apr. 30, 2003
(86) PCT No.: PCT/IB03/02515

(30) Foreign Application Priority Data
Apr. 30, 2002 (GB) 0209893.7

(51) Int. Cl. A61K 38/19; C07K 14/53

(52) U.S. Cl. 424/85.1; 530/351

(57) ABSTRACT

A conjugate of a cytokine and a tumor targeting moiety (TTM) with the provisos that when cytokine is TNF-α, TNF-β or IFN-γ, the TTM is other than a CD13 ligant; when the cytokine is IL-12, the TTM is other than an antiboy to fibronectin; when the cytokine is TNF, the TTM is other than an antibody to the transferrin receptor, and when the cytokine is TNF, IFN-γ, or IL-2 the antibody is other than an antibody to the TAG72 antigen.

### FIELD OF THE INVENTION

The present invention relates to a pharmaceutical composition and uses thereof.

### BACKGROUND OF THE INVENTION

Tumor growth and mass represent the major limiting factor to successful immunotherapies. Surgical, chemio and radiation therapies are conventionally used to debulk tumors, with variable success depending on the localization of the tumor, its diffusion and intrinsic resistance to treatments. In spite of measurable improvement in patients survival, these conventional therapies still presents conspicuous drawbacks. Debulking by surgery may be very efficient in removing the primary tumor mass, but is of limited clinical utility with disseminated metastastatic tumors. On the other hand, chemotherapy may be associated with the risk of selecting resistant variants, which then become untreatable. Furthermore, chemotherapy is generally very toxic for patients, and has strong immunosuppressive effects. For these reasons, it is necessary to develop new approaches for cancer treatment based on different principles, with low toxicity and high efficiency in eradicating disseminated lesions.

The antitumor activity of some cytokines is described. Some cytokines have already been used therapeutically in humans. For example, cytokines such as IL-2 and IFN-γ have shown positive antitumoral activity in patients with different types of tumors, such as kidney metastatic carcinoma, hairy cell leukemia, Kaposi sarcoma, melanoma, multiple mieloma, and the like. Other cytokines like IFNβ, the Tumor Necrosis Factor (TNF)α, TNFβ, IL-1, 4, 6, 12, 15 and the Colony Stimulating Factors (CFSs) have shown a certain antitumoral activity on some types of tumors.

In general, the therapeutic use of cytokines is strongly limited by their systemic toxicity. TNF, for example, was originally discovered for its capacity for inducing the hemorrhagic necrosis of some tumors, and for its in vitro cytotoxic effect on different tumoral lines, but is subsequently proved to have strong pro-inflammatory activity, which can, in case of overproduction conditions, dangerously affect the human body.

As the systemic toxicity is a fundamental problem with the use of pharmacologically active amounts of cytokines in humans, novel derivatives and therapeutic strategies are now under evaluation, aimed at reducing the toxic effects of this class of biological effectors while keeping their therapeutic efficacy.

Some novel approaches are directed to:
a) the development of fusion proteins which can deliver TNF into the tumor and increase the local concentration. For example, the fusion proteins consisting of TNF and tumor specific-antibodies have been produced;
b) the development of TNF mutants which maintain the antitumoral activity and have a reduced systemic toxicity. Accordingly, mutants capable of selectively recognizing only one receptor have already been prepared;
c) the use of anti-TNF antibodies able to reduce some toxic effects of TNF without compromising its antitumoral activity. Such antibodies have already been described in literature;
d) the use of TNF derivatives with a higher half-life (for example TNF conjugated with polyethylene glycol).

EP 251 494 discloses a system for administering a diagnostic or therapeutic agent, which comprises: an antibody conjugated with avidin or streptavidin, an agent capable of complexing the conjugated antibody and a compound consisting of the diagnostic or therapeutic agent conjugated with biotin, which are administered sequentially and adequately delayed, so as to allow the localization of the therapeutic or diagnostic agent through the biotin-streptavidin interaction on the target cell recognized by the antibody. The described therapeutic or diagnostic agents comprise metal chelates, in particular chelates of radionuclides and low molecular weight antitumoral agents such as cis-platinum, doxorubicin, etc.

EP 496 074 discloses a method which provides the sequential administration of a biotinylated antibody, avidin or streptavidin and a biotinylated diagnostic or therapeutic agent. Although cytotoxic agents like ricin are generically mentioned, the application relative to radiolabelled compounds is mostly disclosed.

WO 95/15979 discloses a method for localizing highly toxic agents on cellular targets, based on the administration of a first conjugate comprising the specific target molecule conjugated with a ligand or an anti-ligand followed by the administration of a second conjugate consisting of the toxic agent bound to an anti-ligand or to the ligand.

WO 99/13329 discloses a method for targeting a molecule to tumoral angiogenic vessels, based on the conjugation of said molecule with ligands of NGR receptors. A number of molecules have been suggested as possible candidates, but doxorubicin only is specifically described. No use of ligands of NGR receptors as cytokines vehicles to induce immuno responses is disclosed.

In WO01/61017 the current inventor describes how surprisingly it has been found that the therapeutic index of certain cytokines can be remarkably improved and their immunotherapeutic properties can be enhanced by coupling with a ligand of the aminopeptidase-N receptor (CD13). CD13 is a transmembrane glycoprotein of 150 kDa which is highly conserved in various species. It is expressed on normal cells as well as in myeloid tumor lines, in the angiogenic endothelium and in some epithelia. The CD13 receptor is usually identified as the “NGR” receptor, in that its peptide ligands share the amino acid “NGR” motif.

Halin C et al (2002) Nature Biotechnology 20:264-269 discloses a fusion protein consistiing of IL-12 fused to a human antibody fragment specific to the oncofetal ED-B domain of fibronectin. Carnemolla et al (2002) Blood 99(5):1659-65 discloses a fusion protein of IL-2 and an antibody to ED-B.

Corti A et al (1998) Cancer Research 58:3866-3872 discloses an indirect appoach or “pretargeting” approach to homing TNF to tumors comprising tumor pre-targeting with biotintylated antibodies and avidin or streptavidin, followed by delyaed delivery of biotinylated TNF.

Hoogenboom et al (1991) Mol. Immunol. 28:1027-1037 discloses a fusion protein constructed by fusing part of the heavy chain gene of an anti-transferrin receptor mAb with the TNF-α gene. Yang et al (1995) Hum. Antibod. Hybrodomas 6:129-136 discloses fusing the N-terminus of TNF with the C-terminus of the hinge region of a mAb against the tumor-associated TAG72 antigen expressed by colorectal, gastric and ovarian adenocarcinoma. Yang et al (1995) Mol Immunol 32:873-881 discloses the production of a monovalent Fv-TNF fusion protein with the TAG72 antigen. To our knowledge no data on the in vivo activity of these conjugates has been reported.

Xiang et al (1993) Cancer Biother 8:327-337 discloses a recombinant bifunctional molecule of the single-chain Fv directed to TAG72 and IFN-γ; and Xiang et al (1994) Immun Cell Biol 72:275-285 discloses discloses a recombinant bifunctional molecule of the single-chain Fv directed to TAG72 and IL-2.

However, there remains a need for further and improved pharmaceutical compositions and methods for the treatment and diagnosis of cancer.

We have now found that the concept of targeted delivery of cytokines is broadly applicable and surprisingly increases the therapeutic index of chemotherapeutic drugs. Due to the complexity of the multivalent interactions necessary for these conjugates to work (targeting receptor, TNF receptors) it is not obvious that vascular receptors different from CD13 can work.

Statements of the Invention

According to one aspect of the present invention there is provided a conjugate of a cytokine and a tumor targeting moiety (TTM) with the provisos that when the cytokine is TNF-α, TNF-β or IFN-γ, the TTM is other than a CD13 ligand; when the cytokine is IL-2 or IL-12, the TTM is other than an antibody to fibronectin; when the cytokine is TNF, the TTM is other than an antibody to the transferrin receptor; when the cytokine is TNF, IFN-γ or IL-2 the TTM is other than an antibody to the TAG72 antigen; when the cytokine is IFN, the TTM is other than αvβ₃ integrin ligand; and when the cytokine is TNF, the TTM is other than fibronectin.

In another embodiment the conjugate is not biotinylated TNF.

Preferably the cytokine is an inflammatory cytokine.

In one preferred embodiment the cytokine is a chemotherapeutic cytokine.

Preferably the cytokine is TNFα, TNFβ, IFNα, IFNβ, IFNγ, IL-1,2, 4, 6, 12, 15, EMAP II, vascular endothelial growth factor (VEGF), PDGF, PD-ECGF or a chemokine.

In one embodiment the cytokine is TNF-α, TNF-β or IFN-γ.

The target compound can be expressed either on the endothelial cells surface of tumor vessels or in the extracellular matrix in close contact with or in the vicinity of endothelial cells.

In one embodiment the TTM is a tumor vasculature targeting moiety (TVTM).

In another embodiment the TVTM is a binding partner of a tumor vasculature receptor, marker or other extracellular component.

In another embodiment the TTM is a binding partner of a tumor receptor, marker or other extracellular component.

In another embodiment the TTM is an antibody or ligand, or a fragment thereof. In one embodiment the TTM is contains the NGR or RGD motif, or is HIV-tat, Annexin V, Osteopontin, Fibronectin, Collagen Type I or IV, Hyaluronate, Ephrin, or is a binding partner to oncofetal fibronectin; or a fragment thereof. In one embodiment the TTM is other than HIV-tat.

In a preferred embodiment the TTM contains the NGR motif

Preferably the TTM is CNGRCVSGCAGRC, NGRAHA, GNGRG, cycloCVLNGRMEC, linear or cyclic CNGRC.

In another preferred embodiment the TTM contains the RGD motif.

In one embodiment the TTM is targeted to VEGFR, ICAM 1, 2 or 3, PECAM-1, CD31, CD13, VCAM-1, Selectin, Act R11, ActRIIB, ActRI, ActRIB, CD44, aminopeptidase A, aminopeptidase N (CD13), αvβ3 integrin, αvβ5 integrin, FGF-1, 2, 3, or 4, IL-1R, EPHR, MMP, NG2, tenascin, oncofetal fibronectin, PD-ECGFR, TNFR, PDGFR or PSMA. In another embodiment the TTM is not targeted to VEGFR.

Preferably the conjugate is in the form of a fusion protein.

In another embodiment the conjugate is in the form of nucleic acid.

According to another aspect of the present invention there is provided an expression vector comprising the nucleic acid of the present invention.

According to another aspect of the present invention there is provided a host cell transformed with the expression vector of of the present invention.

According to another aspect of the present invention there is provided a method for preparing a conjugate comprising culturing the host cell of claim under condition which provide for the expression of the conjugate.

According to yet another aspect of the present invention there is provided a pharmaceutical composition comprising the conjugate of the present invention, together with a pharmaceutically acceptable carrier, diliuent or excipient.

In a preferred embodiment the composition further comprises another antitumor agent or diagnostic tumor-imaging compound.

Preferably the further antitumor agent is doxorubicin or melphalan.

According to a further aspect of the present invention there is provided use of a conjugate or a pharmaceutical composition according to the present invention for the preparation of a medicament for treatment or diagnosis of cancer.

Put another way, the present invention provides a method of treating or diagnosing cancer comprising administering to a patient in need of the same an effective amount of a conjugate or a pharmaceutical composition according to the present invention.

Combinations of preferred targeting moieties and cytokines which may be used in the present invention are shown in Table A below.

TABLE A

| Cytokine | Targeting Moiety |
|---|---|
| IFN-α | RGD-CONTAINING PEPTIDE |
| IFN-β | RGD-CONTAINING PEPTIDE |
| IL-2 | RGD-CONTAINING PEPTIDE |
| IL-12 | RGD-CONTAINING PEPTIDE |
| EMAP II | RGD-CONTAINING PEPTIDE |
| VEGF | RGD-CONTAINING PEPTIDE |
| IL-1 | RGD-CONTAINING PEPTIDE |
| IL-6 | RGD-CONTAINING PEPTIDE |
| IL-12 | RGD-CONTAINING PEPTIDE |
| PDGF | RGD-CONTAINING PEPTIDE |
| PD-ECGF | RGD-CONTAINING PEPTIDE |
| CXC chemokine | RGD-CONTAINING PEPTIDE |
| CC chemokine | RGD-CONTAINING PEPTIDE |
| C chemokine | RGD-CONTAINING PEPTIDE |
| IL-15 | RGD-CONTAINING PEPTIDE |
| TNF-α | NGR-CONTAINING PEPTIDE |
| TNF-β | NGR-CONTAINING PEPTIDE |
| IFN-α | NGR-CONTAINING PEPTIDE |
| IFN-β | NGR-CONTAINING PEPTIDE |
| IFN-γ | NGR-CONTAINING PEPTIDE |
| IL-2 | NGR-CONTAINING PEPTIDE |
| IL-12 | NGR-CONTAINING PEPTIDE |
| EMAP II | NGR-CONTAINING PEPTIDE |
| VEGF | NGR-CONTAINING PEPTIDE |
| IL-1 | NGR-CONTAINING PEPTIDE |
| IL-6 | NGR-CONTAINING PEPTIDE |
| IL-12 | NGR-CONTAINING PEPTIDE |
| PDGF | NGR-CONTAINING PEPTIDE |
| PD-ECGF | NGR-CONTAINING PEPTIDE |
| CXC chemokine | NGR-CONTAINING PEPTIDE |
| CC chemokine | NGR-CONTAINING PEPTIDE |
| C chemokine | NGR-CONTAINING PEPTIDE |
| IL-15 | NGR-CONTAINING PEPTIDE |
| TNF-α | Ligand to VEGFR |
| TNF-β | Ligand to VEGFR |
| IFN-α | Ligand to VEGFR |
| IFN-β | Ligand to VEGFR |
| IFN-γ | Ligand to VEGFR |
| IL-2 | Ligand to VEGFR |
| IL-12 | Ligand to VEGFR |
| EMAP II | Ligand to VEGFR |
| VEGF | Ligand to VEGFR |
| IL-1 | Ligand to VEGFR |
| IL-6 | Ligand to VEGFR |
| IL-12 | Ligand to VEGFR |
| PDGF | Ligand to VEGFR |
| PD-ECGF | Ligand to VEGFR |
| CXC chemokine | Ligand to VEGFR |
| CC chemokine | Ligand to VEGFR |
| C chemokine | Ligand to VEGFR |
| IL-15 | Ligand to VEGFR |
| TNF-α | Ab to VEGFR |
| TNF-β | Ab to VEGFR |
| IFN-α | Ab to VEGFR |
| IFN-β | Ab to VEGFR |
| IFN-γ | Ab to VEGFR |
| IL-2 | Ab to VEGFR |
| IL-12 | Ab to VEGFR |
| EMAP II | Ab to VEGFR |
| VEGF | Ab to VEGFR |
| IL-1 | Ab to VEGFR |
| IL-6 | Ab to VEGFR |
| IL-12 | Ab to VEGFR |
| PDGF | Ab to VEGFR |
| PD-ECGF | Ab to VEGFR |
| CXC chemokine | Ab to VEGFR |
| CC chemokine | Ab to VEGFR |
| C chemokine | Ab to VEGFR |
| IL-15 | Ab to VEGFR |
| TNF-α | HIV-tat |
| TNF-β | HIV-tat |
| IFN-α | HIV-tat |
| IFN-β | HIV-tat |
| IFN-γ | HIV-tat |
| IL-2 | HIV-tat |
| IL-12 | HIV-tat |
| EMAP II | HIV-tat |
| VEGF | HIV-tat |
| IL-1 | HIV-tat |
| IL-6 | HIV-tat |
| IL-12 | HIV-tat |
| PDGF | HIV-tat |
| PD-ECGF | HIV-tat |
| CXC chemokine | HIV-tat |
| CC chemokine | HIV-tat |
| C chemokine | HIV-tat |
| IL-15 | HIV-tat |
| TNF-α | Ligand to ICAM 1, 2 or 3 |
| TNF-β | Ligand to ICAM 1, 2 or 3 |
| IFN-α | Ligand to ICAM 1, 2 or 3 |
| IFN-β | Ligand to ICAM 1, 2 or 3 |
| IFN-γ | Ligand to ICAM 1, 2 or 3 |
| IL-2 | Ligand to ICAM 1, 2 or 3 |
| IL-12 | Ligand to ICAM 1, 2 or 3 |
| EMAP II | Ligand to ICAM 1, 2 or 3 |
| VEGF | Ligand to ICAM 1, 2 or 3 |
| IL-1 | Ligand to ICAM 1, 2 or 3 |
| IL-6 | Ligand to ICAM 1, 2 or 3 |
| IL-12 | Ligand to ICAM 1, 2 or 3 |
| PDGF | Ligand to ICAM 1, 2 or 3 |
| PD-ECGF | Ligand to ICAM 1, 2 or 3 |
| CXC chemokine | Ligand to ICAM 1, 2 or 3 |
| CC chemokine | Ligand to ICAM 1, 2 or 3 |
| C chemokine | Ligand to ICAM 1, 2 or 3 |
| IL-15 | Ligand to ICAM 1, 2 or 3 |
| TNF-α | Ab to ICAM 1, 2 or 3 |
| TNF-β | Ab to ICAM 1, 2 or 3 |
| IFN-α | Ab to ICAM 1, 2 or 3 |
| IFN-β | Ab to ICAM 1, 2 or 3 |
| IFN-γ | Ab to ICAM 1, 2 or 3 |
| IL-2 | Ab to ICAM 1, 2 or 3 |
| IL-12 | Ab to ICAM 1, 2 or 3 |
| EMAP II | Ab to ICAM 1, 2 or 3 |
| VEGF | Ab to ICAM 1, 2 or 3 |
| IL-1 | Ab to ICAM 1, 2 or 3 |
| IL-6 | Ab to ICAM 1, 2 or 3 |
| IL-12 | Ab to ICAM 1, 2 or 3 |
| PDGF | Ab to ICAM 1, 2 or 3 |
| PD-ECGF | Ab to ICAM 1, 2 or 3 |
| CXC chemokine | Ab to ICAM 1, 2 or 3 |
| CC chemokine | Ab to ICAM 1, 2 or 3 |
| C chemokine | Ab to ICAM 1, 2 or 3 |
| IL-15 | Ab to ICAM 1, 2 or 3 |
| TNF-α | Ligand to PECAM-1/CD31 |
| TNF-β | Ligand to PECAM-1/CD31 |
| IFN-α | Ligand to PECAM-1/CD31 |
| IFN-β | Ligand to PECAM-1/CD31 |
| IFN-γ | Ligand to PECAM-1/CD31 |
| IL-2 | Ligand to PECAM-1/CD31 |
| IL-12 | Ligand to PECAM-1/CD31 |
| EMAP II | Ligand to PECAM-1/CD31 |
| VEGF | Ligand to PECAM-1/CD31 |
| IL-1 | Ligand to PECAM-1/CD31 |
| IL-6 | Ligand to PECAM-1/CD31 |
| IL-12 | Ligand to PECAM-1/CD31 |
| PDGF | Ligand to PECAM-1/CD31 |
| PD-ECGF | Ligand to PECAM-1/CD31 |
| CXC chemokine | Ligand to PECAM-1/CD31 |
| CC chemokine | Ligand to PECAM-1/CD31 |
| C chemokine | Ligand to PECAM-1/CD31 |
| IL-15 | Ligand to PECAM-1/CD31 |
| TNF-α | Ab to PECAM-1/CD31 |
| TNF-β | Ab to PECAM-1/CD31 |
| IFN-α | Ab to PECAM-1/CD31 |
| IFN-β | Ab to PECAM-1/CD31 |
| IFN-γ | Ab to PECAM-1/CD31 |
| IL-2 | Ab to PECAM-1/CD31 |
| IL-12 | Ab to PECAM-1/CD31 |
| EMAP II | Ab to PECAM-1/CD31 |
| VEGF | Ab to PECAM-1/CD31 |
| IL-1 | Ab to PECAM-1/CD31 |
| IL-6 | Ab to PECAM-1/CD31 |
| IL-12 | Ab to PECAM-1/CD31 |
| PDGF | Ab to PECAM-1/CD31 |
| PD-ECGF | Ab to PECAM-1/CD31 |
| CXC chemokine | Ab to PECAM-1/CD31 |
| CC chemokine | Ab to PECAM-1/CD31 |
| C chemokine | Ab to PECAM-1/CD31 |
| IL-15 | Ab to PECAM-1/CD31 |
| TNF-α | Ligand to VCAM-1 |
| TNF-β | Ligand to VCAM-1 |
| IFN-α | Ligand to VCAM-1 |
| IFN-β | Ligand to VCAM-1 |
| IFN-γ | Ligand to VCAM-1 |
| IL-2 | Ligand to VCAM-1 |
| IL-12 | Ligand to VCAM-1 |
| EMAP II | Ligand to VCAM-1 |
| VEGF | Ligand to VCAM-1 |
| IL-1 | Ligand to VCAM-1 |
| IL-6 | Ligand to VCAM-1 |
| IL-12 | Ligand to VCAM-1 |
| PDGF | Ligand to VCAM-1 |
| PD-ECGF | Ligand to VCAM-1 |
| CXC chemokine | Ligand to VCAM-1 |
| CC chemokine | Ligand to VCAM-1 |
| C chemokine | Ligand to VCAM-1 |
| IL-15 | Ligand to VCAM-1 |
| TNF-α | Ab to VCAM-1 |
| TNF-β | Ab to VCAM-1 |
| IFN-α | Ab to VCAM-1 |
| IFN-β | Ab to VCAM-1 |
| IFN-γ | Ab to VCAM-1 |
| IL-2 | Ab to VCAM-1 |
| IL-12 | Ab to VCAM-1 |
| EMAP II | Ab to VCAM-1 |
| VEGF | Ab to VCAM-1 |
| IL-1 | Ab to VCAM-1 |
| IL-6 | Ab to VCAM-1 |
| IL-12 | Ab to VCAM-1 |
| PDGF | Ab to VCAM-1 |
| PD-ECGF | Ab to VCAM-1 |
| CXC chemokine | Ab to VCAM-1 |
| CC chemokine | Ab to VCAM-1 |
| C chemokine | Ab to VCAM-1 |
| IL-15 | Ab to VCAM-1 |
| TNF-α | Ligand to Selectin |
| TNF-β | Ligand to Selectin |
| IFN-α | Ligand to Selectin |
| IFN-β | Ligand to Selectin |
| IFN-γ | Ligand to Selectin |
| IL-2 | Ligand to Selectin |
| IL-12 | Ligand to Selectin |
| EMAP II | Ligand to Selectin |
| VEGF | Ligand to Selectin |
| IL-1 | Ligand to Selectin |
| IL-6 | Ligand to Selectin |
| IL-12 | Ligand to Selectin |
| PDGF | Ligand to Selectin |
| PD-ECGF | Ligand to Selectin |
| CXC chemokine | Ligand to Selectin |
| CC chemokine | Ligand to Selectin |
| C chemokine | Ligand to Selectin |
| IL-15 | Ligand to Selectin |
| TNF-α | Ab to Selectin |
| TNF-β | Ab to Selectin |
| IFN-α | Ab to Selectin |
| IFN-β | Ab to Selectin |
| IFN-γ | Ab to Selectin |
| IL-2 | Ab to Selectin |
| IL-12 | Ab to Selectin |
| EMAP II | Ab to Selectin |
| VEGF | Ab to Selectin |
| IL-1 | Ab to Selectin |
| IL-6 | Ab to Selectin |
| IL-12 | Ab to Selectin |
| PDGF | Ab to Selectin |
| PD-ECGF | Ab to Selectin |
| CXC chemokine | Ab to Selectin |
| CC chemokine | Ab to Selectin |
| C chemokine | Ab to Selectin |
| IL-15 | Ab to Selectin |
| TNF-α | Ligand to ActRII, ActRIIB, ActRI or |
| | ActRIB |
| TNF-β | Ligand to ActRII, ActRIIB, ActRI or |
| | ActRIB |
| IFN-α | Ligand to ActRII, ActRIIB, ActRI or |
| | ActRIB |
| IFN-β | Ligand to ActRII, ActRIIB, ActRI or |
| | ActRIB |
| IFN-γ | Ligand to ActRII, ActRIIB, ActRI or |
| | ActRIB |
| IL-2 | Ligand to ActRII, ActRIIB, ActRI or |
| | ActRIB |
| IL-12 | Ligand to ActRII, ActRIIB, ActRI or |
| | ActRIB |
| EMAP II | Ligand to ActRII, ActRIIB, ActRI or |
| | ActRIB |
| VEGF | Ligand to ActRII, ActRIIB, ActRI or |
| | ActRIB |
| IL-1 | Ligand to ActRII, ActRIIB, ActRI or |
| | ActRIB |
| IL-6 | Ligand to ActRII, ActRIIB, ActRI or |
| | ActRIB |
| IL-12 | Ligand to ActRII, ActRIIB, ActRI or |
| | ActRIB |
| PDGF | Ligand to ActRII, ActRIIB, ActRI or |
| | ActRIB |
| PD-ECGF | Ligand to ActRII, ActRIIB, ActRI or |
| | ActRIB |
| CXC chemokine | Ligand to ActRII, ActRIIB, ActRI or |
| | ActRIB |
| CC chemokine | Ligand to ActRII, ActRIIB, ActRI or |
| | ActRIB |
| C chemokine | Ligand to ActRII, ActRIIB, ActRI or |
| | ActRIB |
| IL-15 | Ligand to ActRII, ActRIIB, ActRI or |
| | ActRIB |
| TNF-α | Ab to ActRII, ActRIIB, ActRI or ActRIB |
| TNF-β | Ab to ActRII, ActRIIB, ActRI or ActRIB |
| IFN-α | Ab to ActRII, ActRIIB, ActRI or ActRIB |
| IFN-β | Ab to ActRII, ActRIIB, ActRI or ActRIB |
| IFN-γ | Ab to ActRII, ActRIIB, ActRI or ActRIB |
| IL-2 | Ab to ActRII, ActRIIB, ActRI or ActRIB |
| IL-12 | Ab to ActRII, ActRIIB, ActRI or ActRIB |
| EMAP II | Ab to ActRII, ActRIIB, ActRI or ActRIB |
| VEGF | Ab to ActRII, ActRIIB, ActRI or ActRIB |
| IL-1 | Ab to ActRII, ActRIIB, ActRI or ActRIB |
| IL-6 | Ab to ActRII, ActRIIB, ActRI or ActRIB |
| IL-12 | Ab to ActRII, ActRIIB, ActRI or ActRIB |
| PDGF | Ab to ActRII, ActRIIB, ActRI or ActRIB |
| PD-ECGF | Ab to ActRII, ActRIIB, ActRI or ActRIB |
| CXC chemokine | Ab to ActRII, ActRIIB, ActRI or ActRIB |
| CC chemokine | Ab to ActRII, ActRIIB, ActRI or ActRIB |
| C chemokine | Ab to ActRII, ActRIIB, ActRI or ActRIB |
| IL-15 | Ab to ActRII, ActRIIB, ActRI or ActRIB |
| TNF-α | Annexin V |
| TNF-β | Annexin V |
| IFN-α | Annexin V |
| IFN-β | Annexin V |
| IFN-γ | Annexin V |
| IL-2 | Annexin V |
| IL-12 | Annexin V |
| EMAP II | Annexin V |
| VEGF | Annexin V |
| IL-1 | Annexin V |
| IL-6 | Annexin V |
| IL-12 | Annexin V |
| PDGF | Annexin V |
| PD-ECGF | Annexin V |
| CXC chemokine | Annexin V |
| CC chemokine | Annexin V |
| C chemokine | Annexin V |
| IL-15 | Annexin V |
| TNF-α | Ligand to CD44 |
| TNF-β | Ligand to CD44 |
| IFN-α | Ligand to CD44 |
| IFN-β | Ligand to CD44 |
| IFN-γ | Ligand to CD44 |
| IL-2 | Ligand to CD44 |
| IL-12 | Ligand to CD44 |
| EMAP II | Ligand to CD44 |
| VEGF | Ligand to CD44 |
| IL-1 | Ligand to CD44 |
| IL-6 | Ligand to CD44 |
| IL-12 | Ligand to CD44 |
| PDGF | Ligand to CD44 |
| PD-ECGF | Ligand to CD44 |
| CXC chemokine | Ligand to CD44 |
| CC chemokine | Ligand to CD44 |
| C chemokine | Ligand to CD44 |
| IL-15 | Ligand to CD44 |
| TNF-α | Ab to CD44 |
| TNF-β | Ab to CD44 |
| IFN-α | Ab to CD44 |
| IFN-β | Ab to CD44 |
| IFN-γ | Ab to CD44 |
| IL-2 | Ab to CD44 |
| IL-12 | Ab to CD44 |
| EMAP II | Ab to CD44 |
| VEGF | Ab to CD44 |
| IL-1 | Ab to CD44 |
| IL-6 | Ab to CD44 |
| IL-12 | Ab to CD44 |
| PDGF | Ab to CD44 |
| PD-ECGF | Ab to CD44 |
| CXC chemokine | Ab to CD44 |
| CC chemokine | Ab to CD44 |
| C chemokine | Ab to CD44 |
| IL-15 | Ab to CD44 |
| TNF-α | Osteopontin |
| TNF-β | Osteopontin |
| IFN-α | Osteopontin |
| IFN-β | Osteopontin |
| IFN-γ | Osteopontin |
| IL-2 | Osteopontin |
| IL-12 | Osteopontin |
| EMAP II | Osteopontin |
| VEGF | Osteopontin |
| IL-1 | Osteopontin |
| IL-6 | Osteopontin |
| IL-12 | Osteopontin |
| PDGF | Osteopontin |
| PD-ECGF | Osteopontin |
| CXC chemokine | Osteopontin |
| CC chemokine | Osteopontin |
| C chemokine | Osteopontin |
| IL-15 | Osteopontin |
| TNF-α | Fibronectin |
| TNF-β | Fibronectin |
| IFN-α | Fibronectin |
| IFN-β | Fibronectin |
| IFN-γ | Fibronectin |
| IL-2 | Fibronectin |
| EMAP II | Fibronectin |
| VEGF | Fibronectin |
| IL-1 | Fibronectin |
| IL-6 | Fibronectin |
| IL-12 | Fibronectin |
| PDGF | Fibronectin |
| PD-ECGF | Fibronectin |
| CXC chemokine | Fibronectin |
| CC chemokine | Fibronectin |
| C chemokine | Fibronectin |
| IL-15 | Fibronectin |
| TNF-α | Collagen type I or IV |
| TNF-β | Collagen type I or IV |
| IFN-α | Collagen type I or IV |
| IFN-β | Collagen type I or IV |
| IFN-γ | Collagen type I or IV |
| IL-2 | Collagen type I or IV |
| IL-12 | Collagen type I or IV |
| EMAP II | Collagen type I or IV |
| VEGF | Collagen type I or IV |
| IL-1 | Collagen type I or IV |
| IL-6 | Collagen type I or IV |
| IL-12 | Collagen type I or IV |
| PDGF | Collagen type I or IV |
| PD-ECGF | Collagen type I or IV |
| CXC chemokine | Collagen type I or IV |
| CC chemokine | Collagen type I or IV |
| C chemokine | Collagen type I or IV |
| IL-15 | Collagen type I or IV |
| TNF-α | Hyaluronate |
| TNF-β | Hyaluronate |
| IFN-α | Hyaluronate |
| IFN-β | Hyaluronate |
| IFN-γ | Hyaluronate |
| IL-2 | Hyaluronate |
| IL-12 | Hyaluronate |
| EMAP II | Hyaluronate |
| VEGF | Hyaluronate |
| IL-1 | Hyaluronate |
| IL-6 | Hyaluronate |
| IL-12 | Hyaluronate |
| PDGF | Hyaluronate |
| PD-ECGF | Hyaluronate |
| CXC chemokine | Hyaluronate |
| CC chemokine | Hyaluronate |
| C chemokine | Hyaluronate |
| IL-15 | Hyaluronate |
| TNF-α | Ligand to FGF-1, 2, 3 or 4 |
| TNF-β | Ligand to FGF-1, 2, 3 or 4 |
| IFN-α | Ligand to FGF-1, 2, 3 or 4 |
| IFN-β | Ligand to FGF-1, 2, 3 or 4 |
| IFN-γ | Ligand to FGF-1, 2, 3 or 4 |
| IL-2 | Ligand to FGF-1, 2, 3 or 4 |
| IL-12 | Ligand to FGF-1, 2, 3 or 4 |
| EMAP II | Ligand to FGF-1, 2, 3 or 4 |
| VEGF | Ligand to FGF-1, 2, 3 or 4 |
| IL-1 | Ligand to FGF-1, 2, 3 or 4 |
| IL-6 | Ligand to FGF-1, 2, 3 or 4 |
| IL-12 | Ligand to FGF-1, 2, 3 or 4 |
| PDGF | Ligand to FGF-1, 2, 3 or 4 |
| PD-ECGF | Ligand to FGF-1, 2, 3 or 4 |
| CXC chemokine | Ligand to FGF-1, 2, 3 or 4 |
| CC chemokine | Ligand to FGF-1, 2, 3 or 4 |
| C chemokine | Ligand to FGF-1, 2, 3 or 4 |
| IL-15 | Ligand to FGF-1, 2, 3 or 4 |
| TNF-α | Ab to FGF-1, 2, 3 or 4 |
| TNF-β | Ab to FGF-1, 2, 3 or 4 |
| IFN-α | Ab to FGF-1, 2, 3 or 4 |
| IFN-β | Ab to FGF-1, 2, 3 or 4 |
| IFN-γ | Ab to FGF-1, 2, 3 or 4 |
| IL-2 | Ab to FGF-1, 2, 3 or 4 |
| IL-12 | Ab to FGF-1, 2, 3 or 4 |
| EMAP II | Ab to FGF-1, 2, 3 or 4 |
| VEGF | Ab to FGF-1, 2, 3 or 4 |
| IL-1 | Ab to FGF-1, 2, 3 or 4 |
| IL-6 | Ab to FGF-1, 2, 3 or 4 |
| IL-12 | Ab to FGF-1, 2, 3 or 4 |
| PDGF | Ab to FGF-1, 2, 3 or 4 |
| PD-ECGF | Ab to FGF-1, 2, 3 or 4 |
| CXC chemokine | Ab to FGF-1, 2, 3 or 4 |
| CC chemokine | Ab to FGF-1, 2, 3 or 4 |
| C chemokine | Ab to FGF-1, 2, 3 or 4 |
| IL-15 | Ab to FGF-1, 2, 3 or 4 |
| TNF-α | Ligand to IL-1R |
| TNF-β | Ligand to IL-1R |
| IFN-α | Ligand to IL-1R |
| IFN-β | Ligand to IL-1R |
| IFN-γ | Ligand to IL-1R |
| IL-2 | Ligand to IL-1R |
| IL-12 | Ligand to IL-1R |
| EMAP II | Ligand to IL-1R |
| VEGF | Ligand to IL-1R |
| IL-1 | Ligand to IL-1R |
| IL-6 | Ligand to IL-1R |
| IL-12 | Ligand to IL-1R |
| PDGF | Ligand to IL-1R |
| PD-ECGF | Ligand to IL-1R |
| CXC chemokine | Ligand to IL-1R |
| CC chemokine | Ligand to IL-1R |
| C chemokine | Ligand to IL-1R |
| IL-15 | Ligand to IL-1R |
| TNF-α | Ab to IL-1R |
| TNF-β | Ab to IL-1R |
| IFN-α | Ab to IL-1R |
| IFN-β | Ab to IL-1R |
| IFN-γ | Ab to IL-1R |
| IL-2 | Ab to IL-1R |
| IL-12 | Ab to IL-1R |
| EMAP II | Ab to IL-1R |
| VEGF | Ab to IL-1R |
| IL-1 | Ab to IL-1R |
| IL-6 | Ab to IL-1R |
| IL-12 | Ab to IL-1R |
| PDGF | Ab to IL-1R |
| PD-ECGF | Ab to IL-1R |
| CXC chemokine | Ab to IL-1R |
| CC chemokine | Ab to IL-1R |
| C chemokine | Ab to IL-1R |
| IL-15 | Ab to IL-1R |
| TNF-α | Ligand to CD31 |
| TNF-β | Ligand to CD31 |
| IFN-α | Ligand to CD31 |
| IFN-β | Ligand to CD31 |
| IFN-γ | Ligand to CD31 |
| IL-2 | Ligand to CD31 |
| IL-12 | Ligand to CD31 |
| EMAP II | Ligand to CD31 |
| VEGF | Ligand to CD31 |
| IL-1 | Ligand to CD31 |
| IL-6 | Ligand to CD31 |
| IL-12 | Ligand to CD31 |
| PDGF | Ligand to CD31 |
| PD-ECGF | Ligand to CD31 |
| CXC chemokine | Ligand to CD31 |
| CC chemokine | Ligand to CD31 |
| C chemokine | Ligand to CD31 |
| IL-15 | Ligand to CD31 |
| TNF-α | Ab to CD31 |
| TNF-β | Ab to CD31 |
| IFN-α | Ab to CD31 |
| IFN-β | Ab to CD31 |
| IFN-γ | Ab to CD31 |
| IL-2 | Ab to CD31 |
| IL-12 | Ab to CD31 |
| EMAP II | Ab to CD31 |
| VEGF | Ab to CD31 |
| IL-1 | Ab to CD31 |
| IL-6 | Ab to CD31 |
| IL-12 | Ab to CD31 |
| PDGF | Ab to CD31 |
| PD-ECGF | Ab to CD31 |
| CXC chemokine | Ab to CD31 |
| CC chemokine | Ab to CD31 |
| C chemokine | Ab to CD31 |
| IL-15 | Ab to CD31 |
| TNF-α | Ligand to EPHR |
| TNF-β | Ligand to EPHR |
| IFN-α | Ligand to EPHR |
| IFN-β | Ligand to EPHR |
| IFN-γ | Ligand to EPHR |
| IL-2 | Ligand to EPHR |
| IL-12 | Ligand to EPHR |
| EMAP II | Ligand to EPHR |
| VEGF | Ligand to EPHR |
| IL-1 | Ligand to EPHR |
| IL-6 | Ligand to EPHR |
| IL-12 | Ligand to EPHR |
| PDGF | Ligand to EPHR |
| PD-ECGF | Ligand to EPHR |
| CXC chemokine | Ligand to EPHR |
| CC chemokine | Ligand to EPHR |
| C chemokine | Ligand to EPHR |
| IL-15 | Ligand to EPHR |
| TNF-α | Ab to EPHR |
| TNF-β | Ab to EPHR |
| IFN-α | Ab to EPHR |
| IFN-β | Ab to EPHR |
| IFN-γ | Ab to EPHR |
| IL-2 | Ab to EPHR |
| IL-12 | Ab to EPHR |
| EMAP II | Ab to EPHR |
| VEGF | Ab to EPHR |
| IL-1 | Ab to EPHR |
| IL-6 | Ab to EPHR |
| IL-12 | Ab to EPHR |
| PDGF | Ab to EPHR |
| PD-ECGF | Ab to EPHR |
| CXC chemokine | Ab to EPHR |
| CC chemokine | Ab to EPHR |
| C chemokine | Ab to EPHR |
| IL-15 | Ab to EPHR |
| TNF-α | Ephrin |
| TNF-β | Ephrin |
| IFN-α | Ephrin |
| IFN-β | Ephrin |
| IFN-γ | Ephrin |
| IL-2 | Ephrin |
| IL-12 | Ephrin |
| EMAP II | Ephrin |
| VEGF | Ephrin |
| IL-1 | Ephrin |
| IL-6 | Ephrin |
| IL-12 | Ephrin |
| PDGF | Ephrin |
| PD-ECGF | Ephrin |
| CXC chemokine | Ephrin |
| CC chemokine | Ephrin |
| C chemokine | Ephrin |
| IL-15 | Ephrin |
| TNF-α | Ligand to MMP |
| TNF-β | Ligand to MMP |
| IFN-α | Ligand to MMP |
| IFN-β | Ligand to MMP |
| IFN-γ | Ligand to MMP |
| IL-2 | Ligand to MMP |
| IL-12 | Ligand to MMP |
| EMAP II | Ligand to MMP |
| VEGF | Ligand to MMP |
| IL-1 | Ligand to MMP |
| IL-6 | Ligand to MMP |
| IL-12 | Ligand to MMP |
| PDGF | Ligand to MMP |
| PD-ECGF | Ligand to MMP |
| CXC chemokine | Ligand to MMP |
| CC chemokine | Ligand to MMP |
| C chemokine | Ligand to MMP |
| IL-15 | Ligand to MMP |
| TNF-α | Ab to MMP |
| TNF-β | Ab to MMP |
| IFN-α | Ab to MMP |
| IFN-β | Ab to MMP |
| IFN-γ | Ab to MMP |
| IL-2 | Ab to MMP |
| IL-12 | Ab to MMP |
| EMAP II | Ab to MMP |
| VEGF | Ab to MMP |
| IL-1 | Ab to MMP |
| IL-6 | Ab to MMP |
| IL-12 | Ab to MMP |
| PDGF | Ab to MMP |
| PD-ECGF | Ab to MMP |
| CXC chemokine | Ab to MMP |
| CC chemokine | Ab to MMP |
| C chemokine | Ab to MMP |
| IL-15 | Ab to MMP |
| TNF-α | Ligand to NG2 |
| TNF-β | Ligand to NG2 |
| IFN-α | Ligand to NG2 |
| IFN-β | Ligand to NG2 |
| IFN-γ | Ligand to NG2 |
| IL-2 | Ligand to NG2 |
| IL-12 | Ligand to NG2 |
| EMAP II | Ligand to NG2 |
| VEGF | Ligand to NG2 |
| IL-1 | Ligand to NG2 |
| IL-6 | Ligand to NG2 |
| IL-12 | Ligand to NG2 |
| PDGF | Ligand to NG2 |
| PD-ECGF | Ligand to NG2 |
| CXC chemokine | Ligand to NG2 |
| CC chemokine | Ligand to NG2 |
| C chemokine | Ligand to NG2 |
| IL-15 | Ligand to NG2 |
| TNF-α | Ab to NG2 |
| TNF-β | Ab to NG2 |
| IFN-α | Ab to NG2 |
| IFN-β | Ab to NG2 |
| IFN-γ | Ab to NG2 |
| IL-2 | Ab to NG2 |
| IL-12 | Ab to NG2 |
| EMAP II | Ab to NG2 |
| VEGF | Ab to NG2 |
| IL-1 | Ab to NG2 |
| IL-6 | Ab to NG2 |
| IL-12 | Ab to NG2 |
| PDGF | Ab to NG2 |
| PD-ECGF | Ab to NG2 |
| CXC chemokine | Ab to NG2 |
| CC chemokine | Ab to NG2 |
| C chemokine | Ab to NG2 |
| IL-15 | Ab to NG2 |
| TNF-α | Ligand to tenascin |
| TNF-β | Ligand to tenascin |
| IFN-α | Ligand to tenascin |
| IFN-β | Ligand to tenascin |
| IFN-γ | Ligand to tenascin |
| IL-2 | Ligand to tenascin |
| IL-12 | Ligand to tenascin |
| EMAP II | Ligand to tenascin |
| VEGF | Ligand to tenascin |
| IL-1 | Ligand to tenascin |
| IL-6 | Ligand to tenascin |
| IL-12 | Ligand to tenascin |
| PDGF | Ligand to tenascin |
| PD-ECGF | Ligand to tenascin |
| CXC chemokine | Ligand to tenascin |
| CC chemokine | Ligand to tenascin |
| C chemokine | Ligand to tenascin |
| IL-15 | Ligand to tenascin |
| TNF-α | Ab to tenascin |
| TNF-β | Ab to tenascin |
| IFN-α | Ab to tenascin |
| IFN-β | Ab to tenascin |
| IFN-γ | Ab to tenascin |
| IL-2 | Ab to tenascin |
| IL-12 | Ab to tenascin |
| EMAP II | Ab to tenascin |
| VEGF | Ab to tenascin |
| IL-1 | Ab to tenascin |
| IL-6 | Ab to tenascin |
| IL-12 | Ab to tenascin |
| PDGF | Ab to tenascin |
| PD-ECGF | Ab to tenascin |
| CXC chemokine | Ab to tenascin |
| CC chemokine | Ab to tenascin |
| C chemokine | Ab to tenascin |
| IL-15 | Ab to tenascin |
| TNF-α | Ligand to PD-ECGFR |
| TNF-β | Ligand to PD-ECGFR |
| IFN-α | Ligand to PD-ECGFR |
| IFN-β | Ligand to PD-ECGFR |
| IFN-γ | Ligand to PD-ECGFR |
| IL-2 | Ligand to PD-ECGFR |
| IL-12 | Ligand to PD-ECGFR |
| EMAP II | Ligand to PD-ECGFR |
| VEGF | Ligand to PD-ECGFR |
| IL-1 | Ligand to PD-ECGFR |
| IL-6 | Ligand to PD-ECGFR |
| IL-12 | Ligand to PD-ECGFR |
| PDGF | Ligand to PD-ECGFR |
| PD-ECGF | Ligand to PD-ECGFR |
| CXC chemokine | Ligand to PD-ECGFR |
| CC chemokine | Ligand to PD-ECGFR |
| C chemokine | Ligand to PD-ECGFR |
| IL-15 | Ligand to PD-ECGFR |
| TNF-α | Ab to PD-ECGFR |
| TNF-β | Ab to PD-ECGFR |
| IFN-α | Ab to PD-ECGFR |
| IFN-β | Ab to PD-ECGFR |
| IFN-γ | Ab to PD-ECGFR |
| IL-2 | Ab to PD-ECGFR |
| IL-12 | Ab to PD-ECGFR |
| EMAP II | Ab to PD-ECGFR |
| VEGF | Ab to PD-ECGFR |
| IL-1 | Ab to PD-ECGFR |
| IL-6 | Ab to PD-ECGFR |
| IL-12 | Ab to PD-ECGFR |
| PDGF | Ab to PD-ECGFR |
| PD-ECGF | Ab to PD-ECGFR |
| CXC chemokine | Ab to PD-ECGFR |
| CC chemokine | Ab to PD-ECGFR |
| C chemokine | Ab to PD-ECGFR |
| IL-15 | Ab to PD-ECGFR |
| TNF-α | Ligand to TNFR |
| TNF-β | Ligand to TNFR |
| IFN-α | Ligand to TNFR |
| IFN-β | Ligand to TNFR |
| IFN-γ | Ligand to TNFR |
| IL-2 | Ligand to TNFR |
| IL-12 | Ligand to TNFR |
| EMAP II | Ligand to TNFR |
| VEGF | Ligand to TNFR |
| IL-1 | Ligand to TNFR |
| IL-6 | Ligand to TNFR |
| IL-12 | Ligand to TNFR |
| PDGF | Ligand to TNFR |
| PD-ECGF | Ligand to TNFR |
| CXC chemokine | Ligand to TNFR |
| CC chemokine | Ligand to TNFR |
| C chemokine | Ligand to TNFR |
| IL-15 | Ligand to TNFR |
| TNF-α | Ab to TNFR |
| TNF-β | Ab to TNFR |
| IFN-α | Ab to TNFR |
| IFN-β | Ab to TNFR |
| IFN-γ | Ab to TNFR |
| IL-2 | Ab to TNFR |
| IL-12 | Ab to TNFR |
| EMAP II | Ab to TNFR |
| VEGF | Ab to TNFR |
| IL-1 | Ab to TNFR |
| IL-6 | Ab to TNFR |
| IL-12 | Ab to TNFR |
| PDGF | Ab to TNFR |
| PD-ECGF | Ab to TNFR |
| CXC chemokine | Ab to TNFR |
| CC chemokine | Ab to TNFR |
| C chemokine | Ab to TNFR |
| IL-15 | Ab to TNFR |
| TNF-α | Ligand to PDGFR |
| TNF-β | Ligand to PDGFR |
| IFN-α | Ligand to PDGFR |
| IFN-β | Ligand to PDGFR |
| IFN-γ | Ligand to PDGFR |
| IL-2 | Ligand to PDGFR |
| IL-12 | Ligand to PDGFR |
| EMAP II | Ligand to PDGFR |
| VEGF | Ligand to PDGFR |
| IL-1 | Ligand to PDGFR |
| IL-6 | Ligand to PDGFR |
| IL-12 | Ligand to PDGFR |
| PDGF | Ligand to PDGFR |
| PD-ECGF | Ligand to PDGFR |
| CXC chemokine | Ligand to PDGFR |
| CC chemokine | Ligand to PDGFR |
| C chemokine | Ligand to PDGFR |
| IL-15 | Ligand to PDGFR |
| TNF-α | Ab to PDGFR |
| TNF-β | Ab to PDGFR |
| IFN-α | Ab to PDGFR |
| IFN-β | Ab to PDGFR |
| IFN-γ | Ab to PDGFR |
| IL-2 | Ab to PDGFR |
| IL-12 | Ab to PDGFR |
| EMAP II | Ab to PDGFR |
| VEGF | Ab to PDGFR |
| IL-1 | Ab to PDGFR |
| IL-6 | Ab to PDGFR |
| IL-12 | Ab to PDGFR |
| PDGF | Ab to PDGFR |
| PD-ECGF | Ab to PDGFR |
| CXC chemokine | Ab to PDGFR |
| CC chemokine | Ab to PDGFR |
| C chemokine | Ab to PDGFR |
| IL-15 | Ab to PDGFR |
| TNF-α | Ligand to PSMA |
| TNF-β | Ligand to PSMA |
| IFN-α | Ligand to PSMA |
| IFN-β | Ligand to PSMA |
| IFN-γ | Ligand to PSMA |
| IL-2 | Ligand to PSMA |
| IL-12 | Ligand to PSMA |
| EMAP II | Ligand to PSMA |
| VEGF | Ligand to PSMA |
| IL-1 | Ligand to PSMA |
| IL-6 | Ligand to PSMA |
| IL-12 | Ligand to PSMA |
| PDGF | Ligand to PSMA |
| PD-ECGF | Ligand to PSMA |
| CXC chemokine | Ligand to PSMA |
| CC chemokine | Ligand to PSMA |
| C chemokine | Ligand to PSMA |
| IL-15 | Ligand to PSMA |
| TNF-α | Ab to PSMA |
| TNF-β | Ab to PSMA |
| IFN-α | Ab to PSMA |
| IFN-β | Ab to PSMA |
| IFN-γ | Ab to PSMA |
| IL-2 | Ab to PSMA |
| IL-12 | Ab to PSMA |
| EMAP II | Ab to PSMA |
| VEGF | Ab to PSMA |
| IL-1 | Ab to PSMA |
| IL-6 | Ab to PSMA |
| IL-12 | Ab to PSMA |
| PDGF | Ab to PSMA |
| PD-ECGF | Ab to PSMA |
| CXC chemokine | Ab to PSMA |
| CC chemokine | Ab to PSMA |
| C chemokine | Ab to PSMA |
| IL-15 | Ab to PSMA |
| TNF-α | Vitronectin |
| TNF-β | Vitronectin |
| IFN-α | Vitronectin |
| IFN-β | Vitronectin |
| IFN-γ | Vitronectin |
| IL-2 | Vitronectin |
| IL-12 | Vitronectin |
| EMAP II | Vitronectin |
| VEGF | Vitronectin |
| IL-1 | Vitronectin |
| IL-6 | Vitronectin |
| IL-12 | Vitronectin |
| PDGF | Vitronectin |
| PD-ECGF | Vitronectin |
| CXC chemokine | Vitronectin |
| CC chemokine | Vitronectin |
| C chemokine | Vitronectin |
| IL-15 | Vitronectin |
| TNF-α | Laminin |
| TNF-β | Laminin |
| IFN-α | Laminin |
| IFN-β | Laminin |
| IFN-γ | Laminin |
| IL-2 | Laminin |
| IL-12 | Laminin |
| EMAP II | Laminin |
| VEGF | Laminin |
| IL-1 | Laminin |
| IL-6 | Laminin |
| IL-12 | Laminin |
| PDGF | Laminin |
| PD-ECGF | Laminin |
| CXC chemokine | Laminin |
| CC chemokine | Laminin |
| C chemokine | Laminin |
| IL-15 | Laminin |
| TNF-α | Ligand to oncofetal fibronectin |
| TNF-β | Ligand to oncofetal fibronectin |
| IFN-α | Ligand to oncofetal fibronectin |
| IFN-β | Ligand to oncofetal fibronectin |
| IFN-γ | Ligand to oncofetal fibronectin |
| IL-2 | Ligand to oncofetal fibronectin |
| IL-12 | Ligand to oncofetal fibronectin |
| EMAP II | Ligand to oncofetal fibronectin |
| VEGF | Ligand to oncofetal fibronectin |
| IL-1 | Ligand to oncofetal fibronectin |
| IL-6 | Ligand to oncofetal fibronectin |
| IL-12 | Ligand to oncofetal fibronectin |
| PDGF | Ligand to oncofetal fibronectin |
| PD-ECGF | Ligand to oncofetal fibronectin |
| CXC chemokine | Ligand to oncofetal fibronectin |
| CC chemokine | Ligand to oncofetal fibronectin |
| C chemokine | Ligand to oncofetal fibronectin |
| IL-15 | Ligand to oncofetal fibronectin |
| TNF-α | Ab to oncofetal fibronectin |
| TNF-β | Ab to oncofetal fibronectin |
| IFN-α | Ab to oncofetal fibronectin |
| IFN-β | Ab to oncofetal fibronectin |
| IFN-γ | Ab to oncofetal fibronectin |
| EMAP II | Ab to oncofetal fibronectin |
| VEGF | Ab to oncofetal fibronectin |
| IL-1 | Ab to oncofetal fibronectin |
| IL-6 | Ab to oncofetal fibronectin |
| PDGF | Ab to oncofetal fibronectin |
| PD-ECGF | Ab to oncofetal fibronectin |
| CXC chemokine | Ab to oncofetal fibronectin |
| CC chemokine | Ab to oncofetal fibronectin |
| C chemokine | Ab to oncofetal fibronectin |
| IL-15 | Ab to oncofetal fibronectin |

It will be appreciated that in the above Table the term “Ab” represents antibody, and that the antibodies and ligands include fragments thereof.

In particularly preferred embodiments the conjugate comprises TNF-α or TNF-β and an NGR-containing peptide, or TNF-α or TNF-β and an RGD-containing peptide.

In a preferred embodiment the conjugate is in the form of a fusion protein.

In another aspect of the present invention there is provided a pharmaceutical composition comprising an effective amount of a conjugation product of TNF and a first TTM or a polynucleotide encoding the same, and an effevctive amount of IFN-γ and a second TTM or a polynucleotide encoding the same, wherein said first TTM and said secoond TTM compete for different receptors.

Some Key Advantages of the Invention

To reach cancer cells in solid tumors, chemotherapeutic drugs must enter the tumor blood vessels, cross the vessel wall and finally migrate through the interstitium. Heterogeneous tumor perfusion, vascular permeability and cell density, and increased interstitial pressure could represent critical barriers that may limit the penetration of drugs into neoplastic cells distant to from tumor vessels and, consequently, the effectiveness of chemotherapy (1). Strategies aimed at improving drug penetration in tumors are, therefore, of great experimental and clinical interest.

A growing body of evidence suggests that Tumor Necrosis Factor-α (TNF), and inflammatory cytokine endowed with potent anti-tumor activity, could be exploited for this purpose. For example, the addition of TNF to regional isolated limb perfusion with melphalan or doxorubicin has produced higher response rates in patients with extremity soft-tissue sarcomas or melanomas than those obtained with chemotherapeutic drugs alone (2-6). TNF-induced alteration of the endothelial barrier function, reduction of tumor interstitial pressure, increased chemotherapeutic drug penetration and tumor vessel damage are believed to be important mechanisms of the synergy between TNF and chemotherapy (3, 4, 7-10). Unfortunately, systemic administration of TNF is accompanied by prohibitive toxicity, the maximum tolerated dose (8-10 μg/kg) being 10-50 times lower than the estimated effective dose (11, 12). For this reason, systemic administration of TNF has been abandoned and the clinical use of this cytokine is limited to locoregional treatments. Nevertheless, some features of the TNF activity, in particular the selectivity for tumor-associated vessels and the synergy with chemotherapeutic drugs, has continued to nourish hopes as regards the possibility of wider therapeutic applications (13).

The vascular effects of TNF provide the rational for developing a “vascular targeting” strategy aimed at increasing the local efficacy and at enabling systemic administration of therapeutic doses. We have shown recently that targeted delivery of TNF to tumor vessels can be achieved by coupling this protein with the CNGRC peptide, an aminopeptidase N (CD13) ligand that targets the tumor neovasculature (14). In the present work, we have investigated whether vascular targeting with other conjugates could enhance the penetration of chemotherapeutic drugs in tumors and improve their efficacy. In addition, we look at whether vascular targeting with the conjugates can reduce drug-penetration barriers and increase the amount of chemotherapeutics that reach cancer cells.

To reduce tumor cells to a number that can be completely destroyed by anti-tumor effector T cells, we must envisage a way to debulk tumor masses in a way that, unlike chemotherapy, is not immunosuppressive.

In this respect, we believe targeting tumor vessels to kill tumor cells appears to be one of the most promising therapeutic approach for cancer. Tumor-induced vascular endothelium is composed of non-transformed cells, which are therefore not subjected to mutations induced by therapy. Thus, repeated treatments that target tumor vascular endothelium could in principle be administered, without running into the danger of selecting for resistant variants. Second, by destroying a relatively low number of tumor vessels, it may be possible to destroy a huge number of tumor cells, which rely on blood support to thrive.

A biological therapy that impairs the function of tumor-associated vessel and disrupt new vessel formation without causing immunosuppression would be, therefore, a very attractive approach to debulk tumor masses prior immunotherapy or other therapeutic interventiions.

Among the various cytokines and biological response modifiers that can affect tumor vessels as well as the immune system, TNF-α, alone or in combination with interferon gamma and chemotherapy is undoubtedly one of the more potent. The massive haemorragic necrosis and tumor shrinkage that this cytokine can induce within 24 hours in animal tumors is well recognized since its discovery. It is now well established that TNF can disrupt the tumor macro- and microvasculature of metastatic melanomas of the extremities also in patients, when regionally administered at high doses in combination with interferon gamma and melphalan, by isolated limb perfusion. TNF can cause a cascade of events leading to endothelial cell damage, platelet aggregation, intravascular fibrin deposition and coagulation, and culminating in the arrest of the tumor circulation. Remarkaly, normal vessels close to the tumor remain unaffected indicating that TNF can somehow distinguish the vasculature of normal tissues from that of tumors. One attractive possibility is therefore to exploit TNF to induce tumor debulking prior other therapeutic intervention.

Another potential advantage of tumor debulking with TNF over conventional chemotherapeutic agents is that it is not an immunosuppressor, but on the contrary, it is an important activator of the immune response. Indeed TNF can activate antigen presenting cell which in turn are important key mediator of the immune response, as well as a variety of other mechanisms that contribute to an efficient immuneresponse.

Unfortunately, the clinical use of TNF as an anticancer drug has been limited so far to loco-regional treatments because of dose-limiting systemic toxicity and poor therapeutic index.

Soluble, bioactive TNF is a homotrimeric protein that slowly dissociates into inactive, monomeric subunits at picomolar levels (1). Biological activities are induced by trimeric TNF upon interaction with and subsequent homotypic clustering of two distinct cell surface receptors (2) of 55-60 kDa and 75-80 kDa, respectively (p55TNFR and p75TNFR). The p55TNFR is thought to mediate most TNF effects (3, (4, (5, (6, (7), whereas the p75TNFR, due to its higher affinity (K_{d}=0.1×10⁻⁹ M vs. 0.5×10⁻⁹ M for p55TNFR), plays an important role in increasing the local concentration of TNF and in passing the ligand to the p55TNFR (8, (9). Besides these supportive or modulating effects, direct signalling by the p75TNFR can also contribute to several cellular responses, like proliferation of thymocytes, fibroblasts and natural killer cells, GM-CSF secretion (2, (10, (11), and in determining locally restricted responses induced by the endogenous membrane-bound form of TNF (12).

Clinical trials performed to demonstrate anti-tumour efficacy of TNF showed that administration of large, therapeutically effective doses of TNF were accompanied by unacceptably high levels of systemic toxicity, the dose-limiting toxicity being usually hypotension. Therefore, attempts to administer TNF, systemically, to tumour patients, have been essentially discontinued. Nevertheless, the remarkable anti-tumour activity of TNF in some animal models has continued to nourish hopes as regards the possibility of a therapeutic application of TNF in humans. This implied, however, the need to find ways to reduce TNF toxicity upon systemic administration or to deliver TNF with relative or absolute selectivity to the actual therapeutic target—the tumour.

The maximum tolerated dose of bolus TNF (intravenous) in humans is 218-410 μg/m² (28), about 10-fold lower than the effective dose in animals (29). Based on data from murine models it is believed that at least 10 times higher dose is necessary to achieve anti-tumor effects in humans.

One approach that has been pursued in order to exploit antitumour activities of TNF, while avoiding its systemic toxicity, has been regional or local administration. Thus, local administration of TNF has shown promising response rates in Kaposi's sarcoma, plasmacytomas, ovarian adenocarcinomas and various metastatic tumours in the liver (30, (31). As regards regional administration, striking results have been obtained when high doses of TNF were used in combination with melphalan in isolated limb perfusion to treat extremity melanoma and sarcoma. This protocol has allowed to achieve 90-100% complete response rates with tumours undergoing haemorragic necrosis 32), an observation consistent with those from preclinical studies in some experimental animal tumour models.

Although these results are encouraging, the applicability of these approaches is likely to remain limited for two main reasons. First, in most instances where locoregional therapy can be envisaged it is likely that, also in the future, the use of other established means of intervention (e.g. surgery, radiotherapy) will prevail. Second, by definition, malignancies tend do disseminate and it is in this setting, where locoregional therapy is precluded, that the medical need for new therapeutic approaches is most acute. In the first clinical study on hyperthermic isolated-limb perfusion, high response rates were obtained with the unique dose of 4 mg of TNF in combination with melphalan and interferon-γ (32). Other works showed that interferon-γ can be omitted and that even lower doses of TNF can be sufficient to induce a therapeutic response (33, (34). Since also these treatments are not devoid of risk of toxicity (35), the vascular targeting with TNF derivatives may represent an alternative approach to reduce toxic effects also in this setting.

### DETAILED DESCRIPTION

Various preferred features and embodiments of the present invention will now be described by way of non-limiting example.

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc (as well as the complete version Current Protocols in Molecular Biology).

Conjugate

The present invention relates to a conjugate which is a molecule comprising at least one targeting moiety/polypeptide linked to at least cytokine formed through genetic fusion or chemical coupling. By “linked” we mean that the first and second sequences are associated such that the second sequence is able to be transported by the first sequence to a target cell. Thus, conjugates include fusion proteins in which the transport protein is linked to a cytokine via their polypeptide backbones through genetic expression of a DNA molecule encoding these proteins, directly synthesised proteins and coupled proteins in which pre-formed sequences are associated by a cross-linking agent. The term is also used herein to include associations, such as aggregates, of the cytokine with the targeting protein. According to one embodiment the second sequence may comprise a polynucleotide sequence. This embodiment may be seen as a protein/nucleic acid complex.

The second sequence may be from the same species as the first sequence, but is present in the conjugate of the invention in a manner different from the natural situation, or from a different species.

The conjugates of the present invention are capable of being directed to a cell so that an effector function corresponding to the polypeptide sequence coupled to the transport sequence can take place.

The peptide can be coupled directly to the cytokine or indirectly through a spacer, which can be a single amino acid, an amino acid sequence or an organic residue, such as 6-aminocapryl-N-hydroxysuccinimide.

The peptide ligand is preferably linked to the cytokine N-terminus thus minimising any interference in the binding of the modified cytokine to its receptor. Alternatively, the peptide can be linked to amino acid residues which are amido- or carboxylic-bond acceptors, which may be naturally occurring on the molecule or artificially inserted using genetic engineering techniques. The modified cytokine is preferably prepared by use of a cDNA comprising a 5′-contiguous sequence encoding the peptide.

According to a preferred embodiment, there is provided a conjugation product between TNF and the CNGRC sequence in which the amino-terminal of TNF is linked to the CNGRC peptide through the spacer G (glycine).

Cytokines

Drug penetration into neoplastic cells is critical for the effectiveness of solid-tumor chemotherapy. To reach cancer cells in solid tumors, chemotherapeutic drugs must enter the drug blood vessels, cross the vessel wall and finally migrate through the interstitium. Heterogeneous tumor perfusion, vascular permeability and cell density, and increased interstitial pressure may represent critical barriers that may limit the penetration of drugs into neoplastic cells and, consequently, the effectiveness of chemotherapy. Cytokines which have the effect of affecting these factors are therefore useful in the present invention. A non-limiting list of cytokines which may be used in the present invention is: TNFα, TNFβ, IFNα, IFNβ, IFNγ, IL-1,2, 4, 6, 12, 15, EMAP II, vascular endothelial growth factor (VEGF), PDGF, PD-ECGF or a chemokine.

TNF

TNF acts as an inflammatory cytokine and has the effect of inducing alteration of the endothelial barrier function, reducing of tumor interstitial pressure, and increasing chemotherapeutic drug penetration and tumor vessel damage.

The first suggestion that a tumor necrotizing molecule existed was made when it was observed that cancer patients occasionally showed spontaneous regression of their tumors following bacterial infections. Subsequent studies in the 1960s indicated that host-associated (or endogenous) mediators, manufactured in response to bacterial products, were likely responsible for the observed effects. In 1975 it was shown that a bacterially-induced circulating factor had strong anti-tumor activity against tumors implanted in the skin in mice. This factor, designated tumor necrosis factor (TNF), was subsequently isolated, cloned, and found to be the prototype of a family of molecules that are involved with immune regulation and inflammation. The receptors for TNF and the other members of the TNF superfamily also constitute a superfamily of related proteins.

TNF-related ligands usually share a number of common features. These features do not include a high degree of overall amino acid (aa) sequence homology. With the exception of nerve growth factor (NGF) and TNF-beta, all ligands are synthesised as type II transmembrane proteins (extracellular C-terminus) that contain a short cytoplasmic segment (10-80 aa residues) and a relatively long extracellular region (140-215 aa residues). NGF, which is structurally unrelated to TNF, is included in this superfamily only because of its ability to bind to the TNFRSF low affinity NGF receptor (LNGFR). NGF has a classic signal sequence peptide and is secreted. TNF-β, in contrast, although also fully secreted, has a primary structure much more related to type II transmembrane proteins. TNF-β might be considered as a type II protein with a non-functional, or inefficient, transmembrane segment. In general, TNFSF members form trimeric structures, and their monomers are composed of beta-strands that orient themselves into a two sheet structure. As a consequence of the trimeric structure of these molecules, it is suggested that the ligands and receptors of the TNSF and TNFRSF superfamilies undergo “clustering” during signal transduction.

TNF-α Human TNF-α is a 233 aa residue, nonglycosylated polypeptide that exists as either a transmembrane or soluble protein. When expressed as a 26 kDa membrane bound protein, TNF-α consists of a 29 aa residue cytoplasmic domain, a 28 aa residue transmembrane segment, and a 176 aa residue extracellular region. The soluble protein is created by a proteolytic cleavage event via an 85 kDa TNF-alpha converting enzyme (TACE), which generates a 17 kDa, 157 aa residue molecule that normally circulates as a homotrimer.

TNF-β/LT-α: TNF-β, otherwise known as lymphotoxin-α (LT-α) is a molecule whose cloning was contemporary with that of TNF-α. Although TNF-β circulates as a 171 aa residue, 25 kDa glycosylated polypeptide, a larger form has been found that is 194 aa residues long. The human TNF-β cDNA codes for an open reading frame of 205 aa residues (202 in the mouse), and presumably some type of proteolytic processing occurs during secretion. As with TNF-α, circulating TNF-β exists as a non-covalently linked trimer and is known to bind to the same receptors as TNF-α.

In one embodiment the TNF is a mutant form of TNF capable of selectively binding to one of the TNF receptors (Loetscher H et al (1993) J Biol Chem 268:26350-7; Van Ostade X et al (1993) Nature 361:266-9).

Several approaches aimed at reducing systemic toxicity of TNF while preserving its antitumour activity have been pursued. Although the final goal is the same as that in the previous section, i.e. an increase of the therapeutic index, the rationale is significantly different. In the previous case, a generalised enhancement of a single biological activity, cytotoxicity, initially thought to represent an in vitro correlate of the anti-tumour activity of TNF, in the present a selective modification of the biological profile of TNF leading to the preservation of some activities and to the loss of others.

Work along this latter rationale took advantage, mostly, of the possibility to engineer TNF mutants binding to only one of the two TNFR. Efforts in this direction were initiated by the observation that human TNF binds only one (p55TNFR) of the two mouse TNFR, the interaction with the mouse p75TNFR being species-specific (2). In vivo studies showed that systemic toxicity of human TNF was approximately 50 times lower than that of mouse TNF when administered to normal mice, while anti-tumour activity was equivalent (44). These observations suggested that TNF mutants that maintained binding to the p75TNFR might have a more favourable therapeutic index than natural TNF. Indeed, such receptor-selective TNF mutants were subsequently obtained through site-directed mutagenesis approaches (4, (45). Studies performed with a p55TNFR-specific mutant showed that it was as effective as natural TNF with regard to in vivo antitumour activity, whereas activities on neutrophils and endothelial cells, two cell types believed to play an important part in TNF-induced systemic toxicity, were greatly decreased (6).

Although these results were highly encouraging in view of a possible therapeutic use of these mutants in anti-tumour therapy, hopes that had been raised were considerably dampened by the observation that in primates also the p55TNFR plays an important role in systemic toxicity (46) and that the gain in terms of reduced toxicity was lost when the mutants were administered in combination with an agent that increased sensitivity to TNF, like IL-1, LPS or, most importantly in this setting, in the presence of the tumour itself, which sensitises the organism to TNF in a manner similar to that described for the exogenously administered substances previously referred to (47).

In view of the above we teach that coupling these or other TNF muteins with an alpha v beta 3 ligand may result in an improvement of their therapeutic index.

Many other inflammatory cytokines also have the property of increasing endothelial vessel permeability, and it will be appreciated that the invention can be applied to such cytokines, together with agents which increase expression of such cytokines. Inflammatory cytokines, also known as pro-inflammatory cytokines, are a number of polypeptides and glycoproteins with molecular weights between 5 kDa and 70 kDa. They have a stimulating effect on the inflammatory response. The most important inflammatory cytokines are TNF, IL-1, IL-6 and IL-8.

A Table of some cytokines classified as inflammatory cytokines is shown below:

Inflammatory Cytokines

| Group | Individual cytokines |
|---|---|
| Endogenous cytokines | IL-1, TNF-α, IL-6 |
| Up-regulation | IL-1, TNF-α, IL-6, IFN-α, INF-β, |
| | chemokines |
| Stimulation of the production | IL-1, IL-6, IL-11, TNF-α, INF-γ, TGF-β, |
| of acute phase reactants | LIF, OSM, CNTF |
| Chemoattractant cytokines | |
| CXC chemokines | IL-8, PF-4, PBP, NAP-2, β-TG |
| CC chemokines | MIP-1α, MIP-1β, MCP-1, MCP-2, MCP- |
| | 3, RANTES |
| C chemokines | Lymphotactin |
| Stimulation of inflammatory | IL-12 |
| cytokines | |

TGF-β: transforming growth fadtor, LIF: leukemia inhibitory factor; OSM: oncostatin M; CNTF: ciliary neurotrophic factor; PF-4: platelet factor 4; PBP: platelet basic protein; NAP-2: neutrophil activating protein 2; β-TG: β-thromboglobulin; MIP: macrophage inflammatory protein; MCP: monocyte chemoattractant protein.

The up-regulation of inflammatory response is also performed by IL-11, IFN-α, IFN-β, and especially by the members of the chemokine superfamily. TGF-β in some situations has a number of inflammatory activities including chemoattractant effects on neutrophils, T lymphocytes and inactivated monocytes.

IL-2

Because of the central role of the IL-2/IL-2R system in mediation of the immune and inflammatory responses, it is obvious that monitoring and manipulation of this system has important diagnostic and therapeutic implications. IL-2 has shown promise as an anti-cancer drug by virtue of its ability to stimulate the proliferation and activities of tumor-attacking LAK and TIL (tumor-infiltrating lymphocytes) cells. However, problems with IL-2 toxicity are still of concern and merit investigation. The present invention addresses this problem.

IL-15

Interleukin 15 (IL-15) is a novel cytokine that shares many biological properties with, but lacks amino acid sequence homology to, IL-2. IL-15 was originally identified in media conditioned by a monkey kidney epithelial cell line (CVI/EBNA) based on its mitogenic activity on the murine T cell line, CTLL-2. IL-15 was also independently discovered as a cytokine produced by a human adult T cell leukemia cell line (HuT-102) that stimulated T cell proliferation and was designated IL-T. By virtue of its activity as a stimulator of T cells, NK cells, LAK cells, and TILs, IL-2 is currently in clinical trials testing its potential use in treatments for cancer and for viral infections. Because of its similar biological activities, IL-15 should have similar therapeutic potential.

Chemokines

Chemokines are a superfamily of mostly small, secreted proteins that function in leukocyte trafficking, recruiting, and recirculation. They also play a critical role in many pathophysiological processes such as allergic responses, infectious and autoimmune diseases, angiogenesis, inflammation, tumor growth, and hematopoietic development. Approximately 80 percent of these proteins have from 66 to 78 amino acids (aa) in their mature form. The remainder are larger with additional aa occurring upstream of the protein core or as part of an extended C-terminal segment. All chemokines signal through seven transmembrane domain G-protein coupled receptors. There are at least seventeen known chemokine receptors, and many of these receptors exhibit promiscuous binding properties whereby several different chemokines can signal through the same receptor.

Chemokines are divided into subfamilies based on conserved aa sequence motifs. Most family members have at least four conserved cysteine residues that form two intramolecular disulfide bonds. The subfamilies are defined by the position of the first two cysteine residues:
The alpha subfamily, also called the CXC chemokines, have one aa separating the first two cysteine residues. This group can be further subdivided based on the presence or absence of a glu-leu-arg (ELR) aa motif immediately preceding the first cysteine residue. There are currently five CXC-specific receptors and they are designated CXCR1 to CXCR5. The ELR⁺ chemokines bind to CXCR2 and generally act as neutrophil chemoattractants and activators. The ELR− chemokines bind CXCR3 to −5 and act primarily on lymphocytes. At the time of this writing, 14 different human genes encoding CXC chemokines have been reported in the scientific literature with some additional diversity contributed by alternative splicing.
In the beta subfamily, also called the CC chemokines, the first two cysteines are adjacent to one another with no intervening aa. There are currently 24 distinct human beta subfamily members. The receptors for this group are designated CCR1 to CCR11. Target cells for different CC family members include most types of leukocytes.
There are two known proteins with chemokine homology that fall outside of the alpha and beta subfamilies. Lymphotactin is the lone member of the gamma class (C chemokine) which has lost the first and third cysteines. The lymphotactin receptor is designated XCR1. Fractalkine, the only known member of the delta class (CX₃C chemokine), has three intervening aa between the first two cysteine residues. This molecule is unique among chemokines in that it is a transmembrane protein with the N-terminal chemokine domain fused to a long mucin-like stalk. The fractalkine receptor is known as CX₃CR1.
   VEGF

The present invention is also applicable to Vasculature Endothelial Growth Factor (VEGF). Angiogenesis is a process of new blood vessel development from pre-existing vasculature. It plays an essential role in embryonic development, normal growth of tissues, wound healing, the female reproductive cycle (i.e., ovulation, menstruation and placental development), as well as a major role in many diseases. Particular interest has focused on cancer, since tumors cannot grow beyond a few millimeters in size without developing a new blood supply. Angiogenesis is also necessary for the spread and growth of tumor cell metastases.

One of the most important growth and survival factors for endothelium is VEGF. VEGF induces angiogenesis and endothelial cell proliferation and it plays an important role in regulating vasculogenesis. VEGF is a heparin-binding glycoprotein that is secreted as a homodimer of 45 kDa. Most types of cells, but usually not endothelial cells themselves, secrete VEGF. Since the initially discovered VEGF, VEGF-A, increases vascular permeability, it was known as vascular permeability factor. In addition, VEGF causes vasodilatation, partly through stimulation of nitric oxide synthase in endothelial cells. VEGF can also stimulate cell migration and inhibit apoptosis. There are several splice variants of VEGF-A. The major ones include: 121, 165, 189 and 206 amino acids (aa), each one comprising a specific exon addition.

EMAP II

Endothelial-Monocyte Activating Polypeptide-II (EMAP-II) is a cytokine that is an antiangiogenic factor in tumor vascular development, and strongly inhibits tumor growth. Recombinant human EMAP-II is an 18.3 kDa protein containing 166 amino acid residues. EMAP II has also bee found to increase endothelial vessel permeability.

PDGF

It has also been proposed that platelet-derived growth factor (PDGF) antagonists might increase drug-uptake and therapeutic effects of a broad range of anti-tumor agents in common solid tumors. PDGF is a cytokine of 30 kDA and is released by platelets on wounding and stimulates nearby cells to grow and repair the wound.

PD-ECGF

As its name suggests, platelet-derived endothelial cell growth factor (PD-ECGF) was originally isolated from platelets based on its ability to induce mitosis in endothelial cells. Its related protein is gliostatin.

Targeting Moiety

We have found that the therapeutic index of cytokines can be increased by homing of targeting the cytokine to tumor vessels. In addition, since it is known that tumor cells can form part of the lining of tumor vasculature, the present invention encompasses targeting to tumor cells directly as well as to its vasculature. Any convenient tumor or tumor vasculature, particular endothelial cell, targeting moiety may be used in the conjugate of the present invention. Many such targeting moieties are known and these and any which subsequently become available are encompassed within the scope of the present invention. In one embodiment, the targeting moiety is a binding partner, such as a ligand, of a receptor expressed by a tumor cell, or a binding partner, such as an antibody, to a marker or a component of the extracellular matrix associated with tumor cells. More particularly the targeting moiety is binding partner, such as a ligand of, a receptor expressed by tumor-associated vessels, or a binding partner, such as an antibody, to an endothelial marker or a component of the extracellular matrix associated with angiogenic vessels. The term binding partner is used here in its broadest sense and includes both natural and synthetic binding domains, including ligand and antibodies or binding fragments thereof. Thus, said binding partner can be an antibody or a fragment thereof such as Fab, Fv, single-chain Fv, a peptide or a peptido-mimetic, namely a peptido-like molecule capable of binding to the receptor, marker of extracellular component of the cell.

The following represent a non-limiting examples of suitable targeting domains and receptors/markers to which the conjugate may be targeted:

CD13

It has surprisingly been found that the therapeutic index of certain cytokines can be remarkably improved and their immunotherapeutic properties can be enhanced by coupling with a ligand of aminopeptidase-N receptor (CD13). CD13 is a trans-membrane glycoprotein of 150 kDa highly conserved in various species. It is expressed on normal cells as well as in myeloid tumor lines, in the angiogenic endothelium and is some epithelia. CD113 receptor is usually identified as “NGR” receptor, in that its peptide ligands share the amino acidic “NGR” motif The ligand is preferably a straight or cyclic peptide comprising the NGR motif, such as CNGRCVSGCAGRC, NGRAHA, GNGRG, cycloCVLNGRMEC or cycloCNGRC, or more preferably the peptide CNGRC. Further details can be found in our WO01/61017 which is incorporated herein by reference.

TNF Receptor

As with members of the TNF Superfamily, members of the TNF Receptor Superfamily (TNFRSF) also share a number of common features. In particular, molecules in the TNFRSF are all type I (N-terminus extracellular) transmembrane glycoproteins that contain one to six ligand-binding, 40 aa residue cysteine-rich motifs in their extracellular domain. In addition, functional TNFRSF members are usually trimeric or multimeric complexes that are stabilised by intracysteine disulfide bonds. Unlike most members of the TNFSF, TNFRSF members exist in both membrane-bound and soluble forms. Finally, although aa sequence homology in the cytoplasmic domains of the superfamily members does not exceed 25%, a number of receptors are able to transduce apoptotic signals in a variety of cells, suggesting a common function.

CD40: CD40 is a 50 kDa, 277 aa residue transmembrane glycoprotein most often associated with B cell proliferation and differentiation. Expressed on a variety of cell types, human CD40 cDNA encodes a 20 aa residue signal sequence, a 173 aa residue extracellular region, a 22 aa residue transmembrane segment, and a 62 aa residue cytoplasmic domain. There are four cysteine-rich motifs in the extracellular region that are accompanied by a juxtamembrane sequence rich in serines and threonines. Cells known to express CD40 include endothelial cells.

TNFRI/p55/CD120a: TNFRI is a 55 kDa, 455 aa residue transmembrane glycoprotein that is apparently expressed by virtually all nucleated mammalian cells. The molecule has a 190 aa residue extracellular region, a 25 aa residue transmembrane segment, and a 220 aa residue cytoplasmic domain. Both TNF-α and TNF-β bind to TNFRI. Among the numerous cells known to express TNFRI are endothelial cells.

TNFRII/p75/CD120b: Human TNFRII is a 75 kDa, 461 aa residue transmembrane glycoprotein originally isolated from a human lung fibroblast library. This receptor consists of a 240 aa residue extracellular region, a 27 aa residue transmembrane segment and a 173 aa residue cytoplasmic domain. Soluble forms of TNFRII have been identified, resulting apparently from proteolytic cleavage by a metalloproteinase termed TRRE (TNF-Receptor Releasing Enzyme). The shedding process appears to be independent of that for soluble TNFRI. Among the multitude of cells known to express TNFRII are endothelial cells.

CD134L/OX40L: OX40, the receptor for OX40L, is a T cell activation marker with limited expression that seems to promote the survival (and perhaps prolong the immune response) of CD4⁺ T cells at sites of inflammation. OX40L also shows limited expression. Currently only activated CD4⁺, CD8⁺ T cells, B cells, and vascular endothelial cells have been reported to express this factor. The human ligand is a 32 kDa, 183 aa residue glycosylated polypeptide that consists of a 21 aa residue cytoplasmic domain, a 23 aa residue transmembrane segment, and a 139 aa residue extracellular region.

VEGF Receptor Family

There are three receptors in the VEGF receptor family. They have the common properties of multiple IgG-like extracellular domains and tyrosine kinase activity. The enzyme domains of VEGF receptor 1 (VEGF R1, also known as Flt-1), VEGF R2 (also known as KDR or Flk-1), and VEGF R3 (also known as Flt-4) are divided by an inserted sequence. Endothelial cells also express additional VEGF receptors, Neuropilin-1 and Neuropilin-2. VEGF-A binds to VEGF R1 and VEGF R2 and to Neuropilin-1 and Neuropilin-2. P1GF and VEGF-B bind VEGF R1 and Neuropilin-1. VEGF-C and -D bind VEGF R3 and VEGF R2. HIV-tat and peptides derived therefrom have also been found to target the VEGFR.

PDGF Receptors

PDGF receptors are expressed in the stromal compartment in most common solid tumors. Inhibition of stromally expressed PDGF receptors in a rat colon carcinoma model reduces the tumor interstitial fluid pressure and increases tumor transcapillary transport.

PSMA

Prostate specific membrane antigen (PSMA) is also an excellent tumor endothelial marker, and PSMA antibodies can be generated.

Cell Adhesion Molecules (CAMs)

Cell adhesion molecules (CAMs) are cell surface proteins involved in the binding of cells, usually leukocytes, to each other, to endothelial cells, or to extracellular matrix. Specific signals produced in response to wounding and infection control the expression and activation of certain of these adhesion molecules. The interactions and responses then initiated by binding of these CAMs to their receptors/ligands play important roles in the mediation of the inflammatory and immune reactions that constitute one line of the body's defence against these insults. Most of the CAMs characterised so far fall into three general families of proteins: the immunoglobulin (Ig) superfamily, the integrin family, or the selectin family.

A member of the Selectin family of cell surface molecules, L-Selectin consists of an NH2-terminal lectin type C domain, an EGF-like domain, two complement control domains, a 15 amino acid residue spacer, a transmembrane sequence and a short cytoplasmic domain.

Three ligands for L-Selectin on endothelial cells have been identified, all containing O-glycosylated mucin or mucin-like domains. The first ligand, GlyCAM-1, is expressed almost exclusively in peripheral and mesenteric lymph node high endothelial venules. The second L-Selectin ligand, originally called sgp90, has now been shown to be CD34. This sialomucin-like glycoprotein, often used as a surface marker for the purification of pluripotent stem cells, shows vascular expression in a wide variety of nonlymphoid tissues, as well as on the capillaries of peripheral lymph nodes. The third ligand for L-Selectin is MadCAM 1, a mucin-like glycoprotein found on mucosal lymph node high endothelial venules.

P-Selectin, a member of the Selectin family of cell surface molecules, consists of an NH2-terminal lectin type C domain, an EGF-like domain, nine complement control domains, a transmembrane domain, and a short cytoplasmic domain.

The tetrasaccharide sialyl Lewisx (sLex) has been identified as a ligand for both P- and E-Selectin, but P- E- and L-Selectin can all bind sLex and sLea under appropriate conditions. P-Selectin also reportedly binds selectively to a 160 kDa glycoprotein present on murine myeloid cells and to a glycoprotein on myeloid cells, blood neutrophils, monocytes, and lymphocytes termed P-Selectin glycoprotein ligand-1 (PSGL-1), a ligand that also can bind E-Selectin. P-Selectin-mediated rolling of leukocytes can be completely inhibited by a monoclonal antibody specific for PSLG-1, suggesting that even though P-Selectin can bind to a variety of glycoproteins under in vitro conditions, it is likely that physiologically important binding is more limited. A variety of evidence indicates that P-Selectin is involved in the adhesion of myeloid cells, as well as B and a subset of T cells, to activated endothelium.

Ig Superfamily CAMs

The Ig superfamily CAMs are calcium-independent transmembrane glycoproteins. Members of the Ig superfamily include the intercellular adhesion molecules (ICAMs), vascular-cell adhesion molecule (VCAM-1), platelet-endothelial-cell adhesion molecule (PECAM-1), and neural-cell adhesion molecule (NCAM). Each Ig superfamily CAM has an extracellular domain, which contains several Ig-like intrachain disulfide-bonded loops with conserved cysteine residues, a transmembrane domain, and an intracellular domain that interacts with the cytoskeleton. Typically, they bind integrins or other Ig superfamily CAMs. The neuronal CAMs have been implicated in neuronal patterning. Endothelial CAMs play an important role in immune response and inflammation.

In more detail, vascular cell adhesion molecule (VCAM-1, CD106, or INCAM-110), platelet endothelial cell adhesion molecule (PECAM-I/CD31) and intercellular adhesion molecules 1, 2 &3 (ICAM-1, 2 & 3) are five functionally related CAM/IgSF molecules that are critically involved in leukocyte-connective tissue/endothelial cell interactions. Expressed principally on endothelial cells, these molecules in general regulate leukocyte migration across blood vessel walls and provide attachment points for developing endothelium during angiogenesis and are all suitable for targeting in the present invention.

Human CD31 is a 130 kDa, type I (extracellular N-terminus) transmembrane glycoprotein that belongs to the cell adhesion molecule (CAM) or C2-like subgroup of the IgSFl. The mature molecule is 711 amino acid (aa) residues in length and contains a 574 aa residue extracellular region, a 19 aa residue transmembrane segment, and a 118 aa residue cytoplasmic tail. In the extracellular region, there are nine potential N-linked glycosylation sites, and, with a predicted molecular weight of 80 kDa, it appears many of these sites are occupied. The most striking feature of the extracellular region is the presence of six Ig-homology units that resemble the C2 domains of the IgSF. Although they vary in number, the presence of these modules is a common feature of all IgSF adhesion molecules (ICAM-1, 2, 3 & VCAM-1).

Integrins

Integrins are non-covalently linked heterodimers of α and β subunits. To date, 16 α subunits and 8 β subunits have been identified. These can combine in various ways to form different types of integrin receptors. The ligands for several of the integrins are adhesive extracellular matrix (ECM) proteins such as fibronectin, vitronectin, collagens and laminin. Many integrins recognise the amino acid sequence RGD (arginine-glycine-aspartic acid) which is present in fibronectin or the other adhesive proteins to which they bind. Peptides and protein fragments containing the RGD sequence can be used to modulate the activities of the RGD-recognising integrins. Thus the present invention may employ as the targeting moiety peptides recognised by integrins. These peptides are conventionally known as “RGD-containing peptides”. These peptides may include peptides motifs which have been identified as binding to integrins. These motifs include the amino acid sequences: DGR, NGR and CRGDC. The peptide motifs may be linear or cyclic. Such motifs are described in more detail in the following patents which are herein incorporated by reference in relation to their description of an RGD peptides: U.S. Pat. No. 5,536,814 which describes cyclasized CRGDCL, CRGDCA and GACRGDCLGA. U.S. Pat. No. 4,578,079 relates to synthetic peptides of formula X-RGD-T/C—Y where X and Y are amino acids. U.S. Pat. No. 5,547,936 describes a peptide counting the sequence X-RGD-XX where X may be an amino acid. U.S. Pat. No. 4,988,621 describes a number of RGD-counting peptides. U.S. Pat. No. 4,879,237 describes a general peptide of the formula RGD-Y where Y is an amino acid, and the peptide G-RGD-AP. U.S. Pat. No. 5,169,930 describes the peptide RGDSPK which binds to αvβ1 integrin. U.S. Pat. Nos. 5,498,694 and 5,700,908 relate to the cytoplasmic domain of the β3 integrin sub-unit that strictly speaking is not an RGD-containing peptide; although it does contain the sequence RDG. WO97/08203 describes cyclic peptides that are structural mimics or RGD-binding sites. U.S. Pat. No. 5,612,311 describes 15 RGD-containing peptides that are capable of being cyclized either by C-C linkage or through other groups such as penicillamine or mecapto propionic acid analogs. U.S. Pat. No. 5,672,585 describes a general formula encompassing RGD-containing peptides. A preferred group of peptides are those where the aspartic acid residue of RGD is derivatised into an O-methoxy tyrosine derivative. U.S. Pat. No. 5,120,829 describes an RGD cell attachment promoting binding site and a hydrophobic attachment domain. The D form is described in U.S. Pat. No. 5,587,456. U.S. Pat. No. 5,648,330 describes a cyclic RGD-containing peptide that has high affinity for GP Iib/IIIa.

In a preferred embodiment of the present invention the targeting moiety is a ligand for αv β3 or αv β5 integrin.

The use of alpha v beta 3 ligands to convey cytotoxic chemotherapeutic drugs to tumors has been previously reported (WPI 99-215158/199918.). However, in these patent application the idea was to deliver to tumor vessels toxic compounds, such as chemotherapeutic drugs or toxins or anti-angiogenic compounds.

In sharp contrast, TNF is an activator of endothelial and immune cell functions, rather than an inhibitor or a toxic compound. For instance TNF is believed to be a pro-angiogenic molecule and not an anti-angiogenic molecule. Moreover, despite TNF was discovered for its cytotoxicity against some tumor cell lines, it is well known that TNF can seldom kill cells in culture, if protective mechanisms are not blocked, (e.g. with transcription/translation inhibitors).

It would appear therefore that the anti-tumor activity of TNF is based on its activating effects on various cells, and little or not to direct cytotoxic effects on tumor cells or endothelial cells. TNF should be viewed in this context as a biological response modifier and not as a classical cytotoxic compound.

Thus, the therapeutic properties of TNF delivered to alpha v beta 3 are not obvious, simply on the bases of the disclosure of patent WPI 99-215158/199918.

Molecules containing the ACDCRGDCFCG motif are expected to target activated murine as well human vessels (72). Thus, one may expect that human RGD-TNF is endowed with better anti-tumor properties than human TNF in patients, as we observed with the murine counterparts in mice.

The maximum tolerated dose of bolus TNF (intravenous) in humans is 218-410 μg/m² (28), about 10-fold lower than the effective dose in animals (29). Based on data from murine models it is believed that 10-50 times higher dose is necessary to achieve anti-tumor effects in humans (35). In the first clinical study on hyperthermic isolated-limb perfusion, high response rates were obtained with the unique dose of 4 mg of TNF in combination with melphalan and interferon-γ (32). Other works showed that interferon-γ can be omitted and that even lower doses of TNF can be sufficient to induce a therapeutic response (33, (34). Since also these treatments are not devoid of risk of toxicity (35), the use of RGD-TNF may represent an alternative approach to reduce toxic effects at least in this setting.

Moreover, the RGD-TNF cDNA could be used for gene therapy purposes in place of the TNF gene (76) whereas biotinylated RGD-TNF could be applied, in principle, in combination tumor pre-targeting with biotinylated antibodies and avidin (71), to further increase its therapeutic index.

Activin

Cells known to express ActRII include endothelial cells. ActRIIB expression parallels that for ActRII, and is again found in endothelial cells. Cells known to express ActRI include vascular endothelial cells. ActRIB has also been identified in endothelial cells.

Angiogenin

Angiogenin (ANG) is a 14 kDa, non-glycosylated polypeptide so named for its ability to induce new blood vessel growth.

Annexin V

Annexin V is a member of a calcium and phospholipid binding family of proteins with vascular anticoagulant activity. Various synomyms for Annexin V exist: placental protein 4 (PP4), placental anticoagulant protein I (PAP I), calphobindin I (CPB-I), calcium dependent phospholipid binding protein 33 (CaBP33), vascular anticoagulant protein alpha (VACa), anchorin CII, lipocortin-V, endonexin II, and thromboplastin inhibitor. The number of binding sites for Annexin V has been reported as 6-24×106/cell in tumor cells and 8.8×106/cell for endothelial cells.

CD44

Another molecule apparently involved in white cell adhesive events is CD44, a molecule ubiquitously expressed on both hematopoietic and non-hematopoietic cells. CD44 is remarkable for its ability to generate alternatively spliced forms, many of which differ in their activities. This remarkable flexibility has led to speculation that CD44, via its changing nature, plays a role in some of the methods that tumor cells use to progress successfully through growth and metastasis. CD44 is a 80-250 kDa type I (extracellular N-terminus) transmembrane glycoprotein. Cells known to express CD44H include vascular endothelial cells.

There are multiple ligands for CD44, including osteopontin, fibronectin, collagen types I and IV and hyaluronate. Binding to fibronectin is reported to be limited to CD44 variants expressing chrondroitin sulfate, with the chrondroitin sulfate attachment site localised to exons v8-v11. Hyaluronate binding is suggested to be possible for virtually all CD44 isoforms. One of the principal binding sites is proposed to be centred in exon 2 and to involve lysine and arginine residues. Factors other than the simple expression of a known hyaluronate-binding motif also appear to be necessary for hyaluronate binding. Successful hyaluronate binding is facilitated by the combination of exons expressed, a distinctive cytoplasmic tail, glycosylation patterns, and the activity state of the cell. Thus, in terms of its hyaluronate-binding function, a great deal of “potential” flexibility exists within each CD44-expressing cell.

Fibroblast Growth Factor (FGF)

The name “fibroblast growth factor” (FGF) is a limiting description for this family of cytokines. The function of FGFs is not restricted to cell growth. Although some of the FGFs do, indeed, induce fibroblast proliferation, the original FGF molecule (FGF-2 or FGF basic) is now known to also induce proliferation of endothelial cells, chondrocytes, smooth muscle cells, melanocytes, as well as other cells. It can also promote adipocyte differentiation, induce macrophage and fibroblast IL-6 production, stimulate astrocyte migration, and prolong neuronal survival. To date, the FGF superfamily consists of 23 members, all of which contain a conserved 120 amino acid (aa) core region that contains six identical, interspersed amino acids.

FGF-1: Human FGF-1 (also known as FGF acidic, FGFa, ECGF and HBGF-1) is a 17-18 kDa non-glycosylated polypeptide that is expressed by a variety of cells from all three germ layers. The binding molecule may be either an FGF receptor. Cells known to express FGF-1 include endothelial cells.

FGF-2: Human FGF-2, otherwise known as FGF basic, HBGF-2, and EDGF, is an 18 kDa, non-glycosylated polypeptide that shows both intracellular and extracellular activity. Following secretion, FGF-2 is sequestered on either cell surface HS or matrix glycosaminoglycans. Although FGF-2 is secreted as a monomer, cell surface HS seems to dimerize monomeric FGF-2 in a non-covalent side-to-side configuration that is subsequently capable of dimerizing and activating FGF receptors. Cells known to express FGF-2 include endothelial cells.

FGF-3: Human FGF-3 is the product of the int-2 gene [i.e., derived from integration region-2, a region on mouse chromosome 7 that contains a gene (int-2/FGF-3) accidentally activated following retroviral insertion]. The molecule is synthesised as a 28-32 kDa, 222 aa glycoprotein that contains a number of peptide motifs. Cells reported to express FGF-3 are limited to developmental cells and tumors. Tumors known to express FGF-3 include breast carcinomas and colon cancer cell lines.

FGF-4: Human FGF-4 is a 22 kDa, 176 aa glycoprotein that is the product of a developmentally-regulated gene. The molecule is synthesised as a 206 aa precursor that contains a large, ill-defined 30 aa signal sequence plus two heparin-binding motifs (at aa 51-55 and 140-143). The heparin-binding sites directly relate to FGF-4 activity; heparin/heparan regulate the ability of FGF-4 to activate FGFR1 and FGFR2. Cells known to express FGF-4 include both tumor cells and embryonic cells. Its identification in human stomach cancer gives rise to one alternative designation (/hst-1/hst), while its isolation in Kaposi's sarcoma provides grounds for another (K-FGF).

IL-1R

IL-1 exerts its effects by binding to specific receptors. Two distinct IL-1 receptor binding proteins, plus a non-binding signalling accessory protein have been identified. Each have three extracellular immunoglobulin-like (Ig-like) domains, qualifying them for membership in the type IV cytokine receptor family. The two receptor binding proteins are termed type I IL-1 receptor (IL-1 RI) and type II IL-1 receptor (IL-1 RII) respectively. Human IL-1 R1 is a 552 aa, 80 kDa transmembrane glycoprotein that has been isolated from endothelium cells.

RTK

The new family of receptor tyrosine kinase (RTK), the Eph receptors and their ligands ephrins, have been found to be involved in vascular assembly, angiogenesis, tumorigenesis, and metastasis. It has also been that class A Eph receptors and their ligands are elevated in tumor and associated vasculature.

MMP

Matrix metalloproteinases (MMPs) have been implicated in tumor growth, angiogenesis, invasion, and metastasis. They have also been suggested for use as tumor markers.

NG2

NG2 is a large, integral membrane, chondroitin sulfate proteoglycan that was first identified as a cell surface molecule expressed by immature neural cells. Subsequently NG2 was found to be expressed by a wide variety of immature cells as well as several types of tumors with high malignancy. NG2 has been suggested as a target molecule in the tumor vasculature. In particular, collagenase-1 (C1) is the predominant matrix metalloproteinase present in newly formed microvessels and serves as a marker of neovascularization.

Oncofetal Fibronectin

The expression of the oncofetal fragment of fibronectin (Fn-f) has also been found to be increased during angiogenesis and has been suggested as a marker of tumor angiogenesis. In one embodiment the TTM is an antibody or fragment thereof to the oncofetal ED-B domain of fibronectin. The preparation of such an antibody and its conjugation with IL-12 is described in Halin et al (2002) Nature Biotechnology 20:264-269.

Tenascin

Tenascin is a matrix glycoprotein seen in malignant tumors including brain and breast cancers and melanoma. Its expression is malignant but not well differentiated tumors and association with the blood vessels of tumors makes it an important target for both understanding the biology of malignant tumors and angiogenesis, but is a therapeutic cancer target and marker as well.

The targeting moiety is preferably a polypeptide which is capable of binding to a tumor cell or tumor vasculature surface molecule. As well as those mentioned above other such surface molecules which are known or become available may also be targeted by the first sequence.

It will be appreciated that one can apply conventional protein binding assays to identify molecules which bind to surface molecules. It will also be appreciated that one can apply structural-based drug design to develop sequences which bind to surface molecules.

High throughput screening, as described above for synthetic compounds, can also be used for identifying targeting molecules.

This invention also contemplates the use of competitive drug screening assays in which neutralising antibodies capable of binding a target specifically compete with a test compound for binding to a target.

Binding Partner (BP)

The targeting moiety generally take the form of a binding partner (BP) to a surface molecule comprising or consisting of one or more binding domains.

Ligand

The targeting moiety of the present invention may take the form of a ligand. The ligands may be natural or synthetic. The term “ligand” also refers to a chemically modified ligand. The one or more binding domains of the BP may consist of, for example, a natural ligand for a receptor, which natural ligand may be an adhesion molecule or a growth-factor receptor ligand (e.g. epidermal growth factor), or a fragment of a natural ligand which retains binding affinity for the receptor.

Synthetic ligands include the designer ligands. As used herein, the term means “designer ligands” refers to agents which are likely to bind to the receptor based on their three dimensional shape compared to that of the receptor.

Antibodies

Alternatively, the binding domains may be derived from heavy and light chain sequences from an immunoglobulin (Ig) variable region. Such a variable region may be derived from a natural human antibody or an antibody from another species such as a rodent antibody. Alternatively the variable region may be derived from an engineered antibody such as a humanised antibody or from a phage display library from an immunised or a non-immunised animal or a mutagenised phage-display library. As a second alternative, the variable region may be derived from a single-chain variable fragment (scFv). The BP may contain other sequences to achieve multimerisation or to act as spacers between the binding domains or which result from the insertion of restriction sites in the genes encoding the BP, including Ig hinge sequences or novel spacers and engineered linker sequences.

The BP may comprise, in addition to one or more immunoglobulin variable regions, all or part of an Ig heavy chain constant region and so may comprise a natural whole Ig, an engineered Ig, an engineered Ig-like molecule, a single-chain Ig or a single-chain Ig-like molecule. Alternatively, or in addition, the BP may contain one or more domains from another protein such as a toxin.

As used herein, an “antibody” refers to a protein consisting of one or more polypeptides substantially encoded by immunoglobulin genes or fragments of immunoglobulin genes. Antibodies may exist as intact immunoglobulins or as a number of fragments, including those well-characterised fragments produced by digestion with various peptidases. While various antibody fragments are defined in terms of the digestion of an intact antibody, one of skill will appreciate that antibody fragments may be synthesised de novo either chemically or by utilising recombinant DNA methodology. Thus, the term antibody, as used herein also includes antibody fragments either produced by the modification of whole antibodies or synthesised de novo using recombinant DNA methodologies. Antibody fragments encompassed by the use of the term “antibodies” include, but are not limited to, Fab, Fab′, F(ab′)2, scFv, Fv, dsFv diabody, and Fd fragments.

The invention also provides monoclonal or polyclonal antibodies to the surface proteins. Thus, the present invention further provides a process for the production of monoclonal or polyclonal antibodies to polypeptides of the invention.

If polyclonal antibodies are desired, a selected mammal (e.g., mouse, rabbit, goat, horse, etc.) is immunised with an immunogenic polypeptide bearing an epitope(s). Serum from the immunised animal is collected and treated according to known procedures. If serum containing polyclonal antibodies to an epitope contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against binding cell surface epitopes in the polypeptides can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. As mentioned above such fragments include Fv, F(ab′) and F(ab′)₂ fragments, as well as single chain antibodies (scFv). Furthermore, the antibodies and fragments thereof may be humanised antibodies, for example as described in EP-A-239400.

Screens

In one aspect, the invention relates to a method of screening for an agent capable of binding to a tumor or tumor vasculature cell surface molecule, the method comprising contacting the cell surface molecule with an agent and determining if said agent binds to said cell surface molecule.

As used herein, the term “agent” includes, but is not limited to, a compound, such as a test compound, which may be obtainable from or produced by any suitable source, whether natural or not. The agent may be designed or obtained from a library of compounds which may comprise peptides, as well as other compounds, such as small organic molecules and particularly new lead compounds. By way of example, the agent may be a natural substance, a biological macromolecule, or an extract made from biological materials such as bacteria, fungi, or animal particularly mammalian) cells or tissues, an organic or an inorganic molecule, a synthetic test compound, a semi-synthetic test compound, a structural or functional mimetic, a peptide, a peptidomimetics, a derivatised test compound, a peptide cleaved from a whole protein, or a peptides synthesised synthetically (such as, by way of example, either using a peptide synthesizer) or by recombinant techniques or combinations thereof, a recombinant test compound, a natural or a non-natural test compound, a fusion protein or equivalent thereof and mutants, derivatives or combinations thereof.

The agent can be an amino acid sequence or a chemical derivative thereof. The substance may even be an organic compound or other chemical. The agent may even be a nucleotide sequence—which may be a sense sequence or an anti-sense sequence.

Protein

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10 or 20 amino acids.

Polypeptide Homologues

It will be understood that polypeptide sequences for use in the invention are not limited to the particular sequences or fragments thereof but also include homologous sequences obtained from any source, for example related viral/bacterial proteins, cellular homologues and synthetic peptides, as well as variants or derivatives thereof. Polypeptide sequences of the present invention also include polypeptides encoded by polynucleotides of the present invention.

Polypeptide Variants, Derivatives and Fragments

The terms “variant” or “derivative” in relation to the amino acid sequences of the present invention includes any substitution of, variation of, modification of, replacement of, deletion of or addition of one (or more) amino acids from or to the sequence providing the resultant amino acid sequence preferably has targeting activity, preferably having at least 25 to 50% of the activity as the polypeptides presented in the sequence listings, more preferably at least substantially the same activity.

Thus, sequences may be modified for use in the present invention. Typically, modifications are made that maintain the activity of the sequence. Thus, in one embodiment, amino acid substitutions may be made, for example from 1, 2 or 3 to 10, 20 or 30 substitutions provided that the modified sequence retains at least about 25 to 50% of, or substantially the same activity. However, in an alternative embodiment, modifications to the amino acid sequences of a polypeptide of the invention may be made intentionally to reduce the biological activity of the polypeptide. For example truncated polypeptides that remain capable of binding to target molecule but lack functional effector domains may be useful.

In general, preferably less than 20%, 10% or 5% of the amino acid residues of a variant or derivative are altered as compared with the corresponding region depicted in the sequence listings.

Amino acid substitutions may include the use of non-naturally occurring analogues, for example to increase blood plasma half-life of a therapeutically administered polypeptide (see below for further details on the production of peptide derivatives for use in therapy). Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides of the invention also include fragments of the above mentioned polypeptides and variants thereof, including fragments of the sequences. Preferred fragments include those which include an epitope. Suitable fragments will be at least about 5, e.g. 10, 12, 15 or 20 amino acids in length. They may also be less than 200, 100 or 50 amino acids in length. Polypeptide fragments of the proteins and allelic and species variants thereof may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered.

Proteins of the invention are typically made by recombinant means, for example as described below. However they may also be made by synthetic means using techniques well known to skilled persons such as solid phase synthesis. Various techniques for chemical synthesising peptides are reviewed by Borgia and Fields, 2000, TibTech 18: 243-251 and described in detail in the references contained therein.

Preparation

Methods for preparing CD13 L-IFN conjugates have been described in WO01/61017. For instance interferon gamma can be fused with the CNGRC peptide by genetic engineering or by chemical synthesis. Given the dimeric structure of interferon gamma, conjugates bearing two CNGRC moieties at the N-terminus or the C-terminus are preferable to provide multivalent high avidity interactions.

Using similar methods it is possible to prepare CRGDC-IFN-γ conjugates to be used in combination with CNGRC-TNF.

It would be easy for a man skilled in the art to prepare conjugates of alpha v beta 3-L-TNF with antibody or antibody fragments that target tumor cells, or tumor associated vessels to further increase the homing to tumor of this TNF derivatives. For instance, avb3L-TNF could be coupled with antibodies against tumor associates antigens or against other tumor angiogenic markers, e.g. matrix metalloproteases (57) and vascular endothelial growth factor (58) or directed against components of the extracellular matrix, such as anti-tenascin antibodies or anti-fibronectin EDB domain.

The avb3L-TNF conjugate could be prepared in many ways. For instance the avb3L is an antibody or a fragment of it, preferably of human origin or bearing a humanized scaffold. In the preferred embody of the invention the avb3L is a peptide. For instance one peptide that bind to avb3 has been recently discovered using phage-peptide libraries. This peptide is characterized by the presence of the sequence CRGDC. Peptides or antibodies can be coupled to TNF using well known recombinant DNA technologies or by chemical conjugation. These molecules could also be prepared by indirect conjugation: for instance they can be both biotinylated and coupled using tetravalent avidin as non covalent cross-linker.

Therapeutic Peptides

Peptides of the present invention may be administered therapeutically to patients. It is preferred to use peptides that do not consisting solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify peptides for therapeutic application. One approach is to link the peptides or proteins to a variety of polymers, such as polyethylene glycol (PEG) and polypropylene glycol (PPG)—see for example U.S. Pat. Nos. 5,091,176, 5,214,131 and U.S. Pat. No. 5,264,209.

Replacement of naturally-occurring amino acids with a variety of uncoded or modified amino acids such as D-amino acids and N-methyl amino acids may also be used to modify peptides.

Another approach is to use bifunctional crosslinkers, such as N-succinimidyl 3-(2 pyridyldithio)propionate, succinimidyl 6-[3-(2 pyridyldithio)propionamido]hexanoate, and sulfosuccinimidyl 6-[3-(2 pyridyldithio)propionamido]hexanoate (see U.S. Pat. No. 5,580,853).

It may be desirable to use derivatives of the peptides of the invention which are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide may be stabilised by a covalent modification, such as cyclization or by incorporation of gamma-lactam or other types of bridges. For example, side chains can be cyclized to the backbone so as create a L-gamma-lactam moiety on each side of the interaction site. See, generally, Hruby et al., “Applications of Synthetic Peptides,” in Synthetic Peptides: A User's Guide: 259-345 (W. H. Freeman & Co. 1992). Cyclization also can be achieved, for example, by formation of cysteine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken. See Wood and Wetzel, 1992, Int'l J. Peptide Protein Res. 39: 533-39.

Another approach described in U.S. Pat. No. 5,891,418 is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic, however a linear construct is typically preferred. One example of a small linear peptide is Gly-Gly-Gly-Gly which has four nitrogens (an N₄ complexation system) in the back bone that can complex to a metal ion with a coordination number of four.

A further technique for improving the properties of therapeutic peptides is to use non-peptide peptidomimetics. A wide variety of useful techniques may be used to elucidating the precise structure of a peptide. These techniques include amino acid sequencing, x-ray crystallography, mass spectroscopy, nuclear magnetic resonance spectroscopy, computer-assisted molecular modelling, peptide mapping, and combinations thereof. Structural analysis of a peptide generally provides a large body of data which comprise the amino acid sequence of the peptide as well as the three-dimensional positioning of its atomic components. From this information, non-peptide peptidomimetics may be designed that have the required chemical functionalities for therapeutic activity but are more stable, for example less susceptible to biological degradation. An example of this approach is provided in U.S. Pat. No. 5,811,512.

Techniques for chemically synthesising therapeutic peptides of the invention are described in the above references and also reviewed by Borgia and Fields, 2000, TibTech 18: 243-251 and described in detail in the references contained therein.

Bifunctional Derivatives

A further embodiment of the invention is provided by bifunctional derivatives in which the cytokines modified with a TTM are conjugated with antibodies, or their fragments, against tumoral antigens or other tumor angiogenic markers, e.g. αv integrins, metalloproteases or the vascular growth factor, or antibodies or fragments thereof directed against components of the extracellular matrix, such as anti-tenascin antibodies or anti-fibronectin EDB domain. The preparation of a fusion product between TNF and the hinge region of a mAb against the tumor-associated TAG72 antigen expressed by gastric and ovarian adenocarcinoma has recently been reported.

A further embodiment of the invention is provided by the tumoral pre-targeting with the biotin/avidin system. According to this approach, a ternary complex is obtained on the tumoral antigenic site, at different stages, which is formed by 1) biotinylated mAb, 2) avidin (or streptavidin) and 3) bivalent cytokine modified with the TTM and biotin. A number of papers proved that the pre-targeting approach, compared with conventional targeting with immunoconjugates, can actually increase the ratio of active molecule homed at the target to free active molecule, thus reducing the treatment toxicity. This approach produced favorable results with biotinylated TNF, which was capable of inducing cytotoxicity in vitro and decreasing the tumor cells growth under conditions in which normal TNF was inactive. The pre-targeting approach can also be carried out with a two-phase procedure by using a bispecific antibody which at the same time binds the tumoral antigen and the modified cytokine. The use of a bispecific antibody directed against a carcinoembryonic antigen and TNF has recently been described as a means for TNF turmoral pre-targeting.

Tumour pre-targeting is another approach that as been recently developed. Pre-targeting can be performed with a variety of different classes of compounds according to a “two-step” or “three-step” approach (59). A specific example based on the avidin-biotin system applied to the radioimmunoscintigraphy of tumours may be helpful in illustrating the principle. In this case, a biotinylated mAb specific for a tumour-associated antigen is administered first (the “targeting” molecule, first step). This is followed one day later by the administration of avidin or streptavidin (the “chase” molecule, second step), tetravalent macromolecules that complex the biotinylated mAb and promote the rapid removal of excess circulating molecules. Another day later radionuclide-labeled biotin (the “effector” molecule, third step) is administered. This is at a time when both the “targeting” and “chase” macromolecules have been efficiently cleared from the circulation. This enables rapid diffusion and localization of the effector to the tumour as well as rapid excretion of excess, circulating free molecules. This is in clear contrast to directly labeled mAb which circulate for significantly longer periods of time thereby increasing backgrounds in radio-immunoscintigraphy and toxic side effects in radio-immunotherapy. Several reports have shown that the pre-targeting approach can indeed greatly improve the target-to-blood ratio compared to conventional targeting with immuno-conjugates and decrease the toxicity of the treatment (60, (61, (62, (63).

Application of the pre-targeting strategy to tumour therapy with biotinylated TNF was considered to be of particular interest because of the markedly higher affinity of the biotin-avidin interaction (10⁻¹⁵M) compared to that of TNF-TNFR interactions. This was expected to allow an efficient, preferential binding of biotinylated TNF to pre-targeted cells over cells expressing TNFR and to prolong its persistence at the tumour site. On the basis of this rationale, the use of a three-step mAb/avidin system for the targeting of biotinylated TNF has been recently described [Moro, 1997]. Mouse RMA lymphoma cells that had been transfected with the Thy 1.1 allele to create a unique turn Gasparri et al 71). A similar approach could be exploited to further increase the therapeutic index of biotinylated avb3L-TNF.

The avb3L-TNF pre-targeting strategy is not necessarily limited to a “three-step” approach. An example of a “two-step” approach, described in the literature, is based on the use of a bispecific antibody with one arm specific for a tumour antigen and with the other for TNF. In particular, it has been recently described the use of a bispecific antibody directed against carcinoembryonic antigen and TNF to target TNF to tumours (64).

According to a further embodiment, the invention comprises a cytokine conjugated to both a TTM and an antibody, or a fragment thereof (directly or indirectly via a boitin-avidin bridge), on different TNF subunits, where the antibody or its fragments are directed against an antigen expressed on tumor cells or other components of the tumor stroma, e.g. tenacin and fibronectin EDB domain. This results in a further improvement of the tumor homing properties of the modified cytokine and in the slow release of the latter in the tumor microenvironment through trimer-monomer-trimer transitions. The modified subunits of e.g. TNF conjugates can disassociate from the targeting complexes and reassociate so as to form unmodified trimeric TNF molecules, which then diffuse in the tumor microenvironment. The release of bioactive TNF has been shown to occur within 24-48 hours after targeting.

The preparation of cytokines in the form of liposomes can improve the biological activity thereof. It has, in fact, been observed that acylation of the TNF amino groups induces an increase in its hydrophobicity without loss of biological activity in vitro. Furthermore, it has been reported that TNF bound to lipids has unaffected cytotoxicity in vitro, immunomodulating effects and reduced toxicity in vivo.

Encapsulation of alpha v beta 3 L-TNF in liposomes could be another way to improve, in qualitative terms, its biological profile. The feasibility of this approach was suggested by the observation that acylation of some amino groups of TNF leads to an increase of its hydrophobicity without loss of biological activity in vitro. This finding has been exploited to easily integrate TNF into lipid vesicles. Such lipid-bound TNF has been reported to possess unchanged in vitro cytotoxicity on tumour cells and immunomodulatory effects, while having less toxic effects in vivo (48, (49).

Derivatisation of alphav beta3L-TNF with polyethylene glycol (pegylation) could be considered a preferred choice for prolonging its half life.

In many instances, the measured half-life of TNF in vivo, may be more apparent than real. Thus, it was observed that this parameter is highly dependent on the administered dose and a disproportionate prolongation of half-life was observed at increasing doses of TNF (50). One explanation for this phenomenon is that, at low doses, TNF is efficiently bound by soluble, circulating TNFR (51). Such soluble TNFR increase rapidly in the serum of patients systemically treated with TNF (52) and arise by proteolytic cleavage from surface-bound receptors. TNF bound to circulating TNFR may escape detection in most assays commonly used for the measurement of TNF levels. Above a threshold level at which all soluble TNFR, both basal as well as TNF-induced, become saturated, measurements start to detect unbound, circulating TNF thereby reflecting, more accurately, the effective in vivo half-life of TNF.

It is clear that pegylation of TNF is not expected to obviate this scavenging effect of TNFR and, thus, any approach aimed at prolonging the half-life of TNF and, more generally, at reducing the doses of TNF to be administered, must deal with the fact that, in order to be active, TNF levels in vivo have to exceed the binding capacity of soluble, circulating TNFR. However one possibility to cope with this problem is to mutagenize CD13L-TNF to reduce its ability to interact with natural TNF receptors, thus enablig higher doses to be administered.

Combined Approach

One of the earliest approaches that has been pursued to achieve a more favourable therapeutic index for systemically administered TNF has been to combine TNF with other agents. The hope was to end up with therapeutic protocols allowing to administer lower doses of TNF which, while preserving anti-tumour activity, had less systemic toxic effects. This rationale was highly speculative because it was not possible to exclude that such protocols would have ended up with a synergistic effect also as regards toxicity and, therefore, with a therapeutic index identical to that observed with TNF alone. In fact, in all instances in which such combination therapy protocols have been studied in humans, it is the latter situation that has proven to be true.

One of these approaches that has been studied most intensively, is the combined use of TNF and IFN-γ (36, 37), particularly because of the synergism of action on endothelial cells of these cytokines. The second approach is the combination with chemotherapy.

Protocols combining TNF and some of the compounds described to synergise with TNF have been studied in some experimental tumour models. Unfortunately, this treatment was accompanied by increased systemic toxicity.

Targeted delivery of TNF to tumor vessels is an approach that has been recently pursued to increase the therapeutic index of TNF. WO01/61017 describes a TNF derivative with improved therapeutic index prepared by coupling TNF with a ligand of aminopeptidase N (CD13), a membrane preotease expressed in tumor vessels. This cytokine interacts in a very complex manner with CD13 and TNF-receptors to selectively activate at low doses tumor endothelial cells. Given the synergistic effect of TNF and IFN-γ on endothelial cells it would be advisable to target endothelial cells with both cytokines conjugated to CD 13 ligands. However, one might expect that these modified cytokines compete for the same receptor (CD13) on endothelial cells leading to loss of targeting and activity. WO01/61017 teaches how to prepare conjugates of this cytokine with CD13 ligands, e.g. NGR-TNF and NGR-IFN-γ. Experiments carried out in our laboratory based on administration of TNF and IFN-γ both conjugated to a CD13 ligand (CNGRC) showed that indeed when these modified cytokines are injected in animal models their therapeutic activity is lower than when given alone, presumably because they compete for the same targeting receptor.

We have now found that these cytokines can be targeted to vessels without cross-interference in binding by, for example, targeting TNF to a tumor vascular receptor different to CD13 and IFN-γ to CD13 (e.g. by coupling it to CNGRC peptide) or vice versa.

In this preferred embodiment of the invention, TNF is coupled to ligands of alpha v beta 3, such as peptides containing the CRGDC motif Thus in one preferred embodiment of the present invention there is provided the combined use of avb3L-IFN-γ derivative with CD13 ligand-TNF. In another preferred embodiment there is provided the combined use of avb3L-TNF derivative with CD13 ligand-IFN-γ.

Polynucleotides

Polynucleotides for use in the invention comprise nucleic acid sequences encoding the polypeptide conjugate of the invention. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

Nucleotide Vectors

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for certain cells may also be used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Host Cells

Vectors and polynucleotides of the invention may be introduced into host cells for the purpose of replicating the vectors/polynucleotides and/or expressing the proteins of the invention encoded by the polynucleotides of the invention. Although the proteins of the invention may be produced using prokaryotic cells as host cells, it is preferred to use eukaryotic cells, for example yeast, insect or mammalian cells, in particular mammalian cells.

Vectors/polynucleotides of the invention may introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

Administration

Proteins of the invention may preferably be combined with various components to produce compositions of the invention. Preferably the compositions are combined with a pharmaceutically acceptable carrier, diluent or excipient to produce a pharmaceutical composition (which may be for human or animal use). Suitable carriers and diluents include isotonic saline solutions, for example phosphate-buffered saline. Details of excipients may be found in The Handbook of Pharmaceutical Excipients, 2nd Edn, Eds Wade & Weller, American Pharmaceutical Association. The composition of the invention may be administered by direct injection. The composition may be formulated for parenteral, intramuscular, intravenous, subcutaneous, intraocular, oral or transdermal administration.

The conjugate may typically be administered in a doasge of about 1 to 10 mg.

The composition may be formulated such that administration daily, weekly or monthly will provide the desired daily dosage. It will be appreciated that the composition may be conveniently formulated for administrated less frequently, such as every 2, 4, 6, 8, 10 or 12 hours.

Polynucleotides/vectors encoding polypeptide components may be administered directly as a naked nucleic acid construct, preferably further comprising flanking sequences homologous to the host cell genome.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector of the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosage regimens described are intended only as a guide since a skilled practitioner will be able to determine readily the optimum route of administration and dosage regimens for any particular patient and condition.

Viral Vectors

In a preferred embodiment the conjugate is administered using a viral vector, more preferably a retroviral vector.

Retroviruses

The retroviral vector for use the present invention may be derived from or may be derivable from any suitable retrovirus. A large number of different retroviruses have been identified. Examples include: murine leukemia virus (MLV), human immunodeficiency virus (HIV), simian immunodeficiency virus, human T-cell leukemia virus (HTLV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “retroviruses”, Cold Spring Harbour Laboratory Press Eds: J M Coffin, S M Hughes, H E Varmus pp 758-763.

Details on the genomic structure of some retroviruses may be found in the art. By way of example, details on HIV and Mo-MLV may be found from the NCBI Genbank (Genome Accession Nos. AF033819 and AF033811, respectively).

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

The lentivirus group can be split even further into “primate” and “on-primate”. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

This invention also relates to the use of vectors for the delivery of a conjugate in the form of a nucleotide sequence to a haematopoietic stem cell (HSC).

Gene transfer involves the delivery to target cells, such as HSCs, of an expression cassette made up of one or more nucleotide sequences and the sequences controlling their expression. This can be carried out ex vivo in a procedure in which the cassette is transferred to cells in the laboratory and the modified cells are then administered to a recipient. Alternatively, gene transfer can be carried out in vivo in a procedure in which the expression cassette is transferred directly to cells within an individual. In both strategies, the transfer process is usually aided by a vector that helps deliver the cassette to the appropriate intracellular site.

Bone marrow has been the traditional source of HSCs for transduction, more recent studies have suggested that peripheral blood stem cells or cord blood cells may be equally good or better target cells (Cassel et al 1993 Exp Hematol 21: 585-591; Bregni et al 1992 Blood 80: 1418-1422; Lu et al 1993 J Exp Med 178: 2089-2096).

Further Anticancer Agents

The conjugate of the present invention may be used in combination with one or more other active agents, such as one or more cytotoxic drugs. Thus, in one aspect of the present invention the method further comprises administering another active pharmaceutical ingredient, such as a cytotoxic drug, either in combined dosage form with the conjugate or in a separate dosage form. Such separate cytotoxic drug dosage form may include solid oral, oral solution, syrup, elixir, injectable, transdermal, transmucosal, or other dosage form. The conjugate and the other active pharmaceutical ingredient can be combined in one dosage form or supplied in separate dosage forms that are usable together or sequentially.

Examples of cytotoxic drugs which may be used in the present invention include: the alkylating drugs, such as cyclophosphamide, ifospfamide, chlorambucil, melphalan, busulfan, lomustine, carmustine, chlormethhine (mustine), estramustine, treosulfan, thiotepa, mitobronitol; cytotoxic antibiotics, such as doxorubicin, epirubicin, aclarubicin, idarubicin, daunorubicin, mitoxantrone (mitozantrone), bleomycin, dactinomycin and mitomycin; antimetabolites, such as methotrexate, capecitabine, cytarabine, fludarabine, cladribine, gemcitabine, fluorouracil, raltitrexed, mercaptopurine, tegafur and tioguanine; vinca alkaloids, such as vinblastine, vincristine, vindesine and vinorelbine, and etoposide; other neoplastic drugs, such as amsacrine, altretamine, crisantaspase, dacarbazine and temozolomide, hydroxycarbamide (hydroxyurea), pentostatin, platinum compounds including: carboplatin, cisplatin and oxaliplatin, porfimer sodium, procarbazine, razoxane, taxanes including: docetaxel and paclitaxel, topoisomerase I inhibitors including: irinotecan and topotecan, trastuzumab, and tretinoin.

In a preferred embodiment the further cytotoxic drug is doxorubicin or melphalan.

The conjugate of the present invention can also be used to use the permeability of tumor cells and vessels to compounds for diagnostic purposes. For instance, the conjugate can be used to increase the tumor uptake of radiolabelled antibodies or hormones (tumor-imaging compounds) in radioimmunoscintigraphy or radiotherapy of tumors.

### FIGURES AND EXAMPLES

The present invention will further be described by reference to the following non-limiting Examples and Figure in which:

FIG. 1 illustrates the characterization of the therapeutic and toxic activity of TNF and RGD-TNF in combination with NGR-IFN in the T/SA mouse mammary adenocarcinoma model. In more detail it shows that the antitumor activity of RGD-mTNF in combination with NGR-mIFN-γ is stronger than that of mTNF administered in combination with NGR-mIFN-γ or that of NGR-mIFN-γ alone. These results indicate that targeted delivery of TNF and IFN-γ to different receptors on the tumor vasculature can produce synergistic effects.

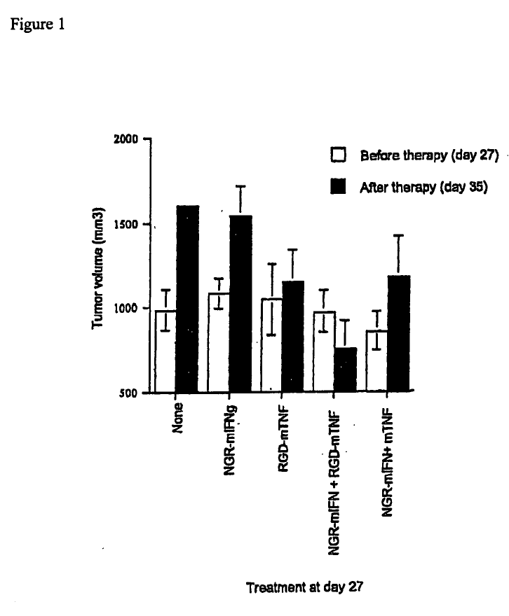

### EXAMPLES

### Example I

Preparation of TNF and RGD-TNF.

Murine recombinant TNF and ACDCRGDCFCG-TNF (RGD-TNF) were produced by cytoplasmic cDNA expression in E. coli. The cDNA coding for murine Met-TNF₁₋₁₅₆ (66) was prepared by reverse transcriptase-polymerase chain reaction (RT-PCR) on mRNA isolated from lipopolysaccharide-stimulated murine RAW-264.7 monocyte-macrophage cells, using 5′-CTGGATCCTCACAGAGCAATGACTCCAAAG-3′ and 5′-TGCCTCACATATGCTCAGATCATCTTCTC-3′, as 3′ and 5′ primers.

The amplified fragment was digested with Nde I and Bam HI (New England Biolabs, Beverley, Mass.) and cloned in pET-I lb (Novagen, Madison, Wis.), previously digested with the same enzymes (pTNF).

The cDNA coding for ACDCRGDCFCG-TNF₁₋₁₅₆ was amplified by PCR on pTNF, using 5′-TGCAGATCATATGGCTTGCGACTGCCGTGGTGACTGCTTCTGCGGTCTCAGAT CATCTTCTC 3′ as 5′ primer, and the above 3′ primer.

The amplified fragment was digested and cloned in pET-11b as described above and used to transform BL21(DE3) E. coli cells (Novagen). The expression of TNF and RGD-TNF was induced with isopropyl-□-D-tiogalactoside, according to the pET11b manufacturer's instruction. Soluble TNF and RGD-TNF were recovered from two-liter cultures by bacterial sonication in 2 mM etilendiaminetetracetic acid, 20 mM Tris-HCl, pH 8.0, followed by centrifugation (15000×g, 20 min, 4° C.). Both extracts were mixed with ammonium sulfate (25% of saturation), left for 1 h at 4° C., and further centrifuged, as above. The ammonium sulfate in the supernatants was then brought to 65% of saturation, left at 4° C. for 24 h and further centrifuged. Each pellet was dissolved in 200 ml of 1 M ammonium sulfate, 50 mM Tris-HCl, pH 8.0, and purified by hydrophobic interaction chromatography on Phenyl-Sepharose 6 Fast Flow (Pharmacia-Upjohn) (gradient elution, buffer A: 50 mM sodium phosphate, pH 8.0, containing 1 M ammonium sulfate; buffer B: 20% glycerol, 5% methanol, 50 mM sodium phosphate, pH 8.0). Fractions containing TNF immunoreactive material (by western blotting) were pooled, dialyzed against 2 mM etilendiaminetetracetic acid, 20 mM Tris-HCl, pH 8.0 and further purified by ion exchange chromatography on DEAE-Sepharose Fast Flow (Pharmacia-Upjohn) (gradient elution, buffer A: 20 mM Tris-HCl, pH 8.0; buffer B: 1 M sodium chloride, 20 mM Tris-HCl, pH 8.0). Fractions containing TNF-immunoreactivity were pooled and purified by gel filtration chromatography on Sephacryl-S-300 HR (Pharmacia-Upjohn), pre-equilibrated and eluted with 150 mM sodium chloride, 50 mM sodium phosphate buffer, pH 7.3 (PBS). Fractions corresponding to 40000-50000 Mr products were pooled, aliquoted and stored frozen at −20° C. All solutions employed in the chromatographic steps were prepared with sterile and endotoxin-free water (Salf, Bergamo, Italy).

The molecular weight of purified TNF and RGD-TNF was measured by electrospray mass spectrometry, as described (65). The protein content was measured using a commercial protein assay kit (Pierce, Rockford, Ill.).

Sodium dodecylsulfate-polyacrylamide gel electrophoresis (SDS-PAGE) and western blot analysis were carried out using 12.5 or 15% polyacrylamide gels, by standard procedures.

Non reducing SDS-PAGE of TNF showed a single band of 17-18 kDa, as expected for monomeric TNF. At variance, non reducing SDS-PAGE and western blot analysis of RGD-TNF showed different immunoreactive forms of 18, 36 and 50 kDa, likely corresponding to monomers, dimers and trimers. Under reducing conditions most of the 50 and 36 kDa bands were converted into the 18 kDa form, pointing to the presence of RGD-TNF molecules with interchain disulfide bridges. The 18 kDa band accounted to about ½ of the total material. These electrophoretic patterns suggest that RGD-TNF was a mixture of trimers made up by three monomeric subunits with correct intra-chain disulfides (10-20%) and the remaining part mostly by trimers with one or more interchain disulfides.

The molecular mass of TNF and RGD-TNF monomers were 17386.1±2.0 Da and 18392.8 Da, respectively, by electrospray mass spectrometry. These values correspond very well to the mass expected for Met-TNF₁₋₁₅₆ (17386.7 Da) and for ACDCRGDCFCG-TNF₁₋₁₅₆ (18392.9 Da).

### Example II

In Vitro Cytotoxic Activity of TNF and RGD-TNF.

The bioactivity of TNF and RGD-TNF was estimated by standard cytolytic assay based on L-M mouse fibroblasts (ATCC CCL1.2) as described (67). The cytolytic activity of TNF and NGR-TNF on RMA-T cells was tested in the presence of 30 ng/ml actinomycin D (68). Each sample was analyzed in duplicate, at three different dilutions. The results are expressed as mean±SD of two-three independent assays.

The in vitro cytotoxic activity of TNF and RGD-TNF was (1.2±0.14)×10⁸ units/mg and (1.7±1)×10⁸ units/mg, respectively, by standard cytolytic assay with L-M cells. These results indicate that the ACDCRGDCFCG moieties in the RGD-TNF molecule does not prevent folding, oligomerizazion and binding to TNF receptors.

In a previous study we showed that RMA-T cells can be killed by TNF in the presence of 30 ng/ml actinomycin D, whereas in the absence of transcription inhibitors these cells are resistant to TNF, even after several days of incubation (68). The in vitro cytotoxic activity of RGD-TNF on RMA-T cells in the presence of actinomycin D was (1.6+1.3)×10⁸ units/mg, as measured using TNF ((1.2±0.14)×10⁸ units/mg) as a standard.

### Example III

Characterization of the Therapeutic and Toxic Activity of TNF and RGD-TNF.

The Rauscher virus-induced RMA lymphoma of C57BL/6 origin (69) were maintained in vitro in RPMI 1640, 5% fetal bovine serum (FBS), 100 U/ml penicillin, 100 μg/ml streptomycin, 0.25 μg/ml amphotericin B, 2 mM glutamine and 50 μM 2-mercaptoethanol. RMA-T was derived from the RMA cell line by transfection with a construct encoding the Thy 1.1 allele and cultured as described Moro, 1997 #28].

T/SA mouse mammary adenocarcinoma cells were cultured as described ( ).

In vivo studies on animal models were approved by the Ethical Committee of the San Raffaele H Scientific Institute and performed according to the prescribed guidelines. C57BL/6 (Charles River Laboratories, Calco, Italy) (16-18 g) were challenged with 5×10⁴ RMA-T or TSA living cells, respectively, s.c. in the left flank. Ten-twelve days after tumor implantation, mice were treated, i.p., with 250 μl TNF or RGD-TNF solutions, diluted with endotoxin-free 0.9% sodium chloride. Preliminary experiments showed that the anti-tumor activity was not changed by the addition of human serum albumin to TNF and RGD-TNF solutions, as a carrier. Each experiment was carried out with 5 mice per group. The tumor growth was monitored daily by measuring the tumor size with calipers. The tumor area was estimated by calculating r₁×r₂ π, whereas tumor volume was estimated by calculating r₁×r₂×r₃×4/3 π, where r₁ and r₂ are the longitudinal and lateral radii, and r₃ is the thickness of tumors protruding from the surface of normal skin. Animals were killed before the tumor reached 1.0-1.3 cm diameter. Tumor sizes are shown as mean±SE (5-10 animals per group) and compared by t-test.

The anti-tumor activity and toxicity of RGD-TNF were compared to those of TNF using the RMA-T lymphoma and the T/SA models in C57BL6 mice.

Murine TNF administered to animals bearing established s.c. RMA-T tumors, causes 24 h later a reduction in the tumor mass and haemorragic necrosis in the central part of the tumor, followed by a significant growth delay for few days (71). A single treatment with TNF does not induce complete regression of this tumor, even at doses close to the LD50, as living cells remaining around the necrotic area restart to grow few days after treatment. In a first set of experiments we investigated the effect of various doses (i.p.) of TNF or RGD-TNF on animal survival. To avoid excessive suffering, the animals were killed when the tumor diameter was greater than 1-1.3 cm. The lethality of TNF and RGD-TNF, 3 days after treatment, was different (LD50, 6 μg and 12 μg, respectively) whereas their anti-tumor activity was markedly different (Table 1). For instance, 1 of μg of RGD-TNF delayed the tumor growth more efficiently then 2 μg of TNF. Interestingly, some animals were cured with 16 μg of RGD-TNF whereas no animals at all were cured with TNF. Cured animals rejected further challenges with tumorigenic doses of either RMA-T or wild-type RMA cells, suggesting that a single treatment with RGD-TNF was able to induce protective immunity.

Thus, the calculated efficacy/toxicity ratio of RGD-TNF under these conditions is 4 times greater than that of TNF. Considering that the form with correct disulfide bridges in the RGD-TNF preparation is about 10-20% one may calculate that the therapeutic index of RGD-TNF is 2040% higher than that of TNF.

Moreover, RGD-TNF can induce protective immune responses more efficiently than TNF.

Since RMA-T cells do not express the alpha v integrin (by FACS with an anti-alpha v antibody) while endothelial cells can express this integrin the results suggest that the mechanism of action is based on targeting cells other than tumor cells, e.g. endothelial cells.

TABLE 1
Survival (%) of RMA-Thy 1.1 lymphoma bearing mice treated 12 days
after tumor implantation with TNF or RGD-TNF (i.v.)

| | | | Survival (%)^{a} | | | | | | |
|---|---|---|---|---|---|---|---|---|---|
| | | | | | | | Day | Day | |
| | Animals | Dose | Day | Day | Day | Day | 90 | 115 | Day |
| Treatment | (n) | (μg i.v.) | 14 | 22 | 32 | 37 | (2nd ch.)^{b} | (3rd ch.)^{b} | 160 |
| none | 9 | 0 | 100 | 0 | | | | | |
| TNF | 9 | 1 | 100 | 22 | 0 | | | | |
| | 8 | 2 | 100 | 37 | 0 | | | | |
| | 10 | 4 | 100 | 70 | 30 | 10 | 0 | | |
| | 10 | 8 | 0 | | | | | | |
| | 10 | 16 | 0 | | | | | | |
| total | 47 | | | | | | | | |
| RGD-TNF | 10 | 1 | 100 | 30 | 20 | 0 | | | |
| | 7 | 2 | 100 | 85 | 15 | 0 | | | |
| | 10 | 4 | 100 | 50 | 10 | 10 | 0 | | |
| | 10 | 8 | 90 | 90 | 30 | 10 | 0 | | |
| | 10 | 16 | 30 | 30 | 20 | 20 | 20 | 20 | 20 |
| total | 47 | | | | | | | | |

^{a}The cumulative results of two independent experiments (5 animals per group of treatment) are shown. Animals with ascitic tumors were not included in the study.
^{b}Surviving animals were re-challanged with 50.000 RMA-T at day 90 followed by 50.000 RMA cells at day 115, respectively. At the same time five normal animals were treated with the same cells to check the tumorigenicity of the injected dose. All control animals developed a tumor within 10 days.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

### REFERENCES

1. Corti A, et al. Biochemical Journal. 1992; 284: 905-10.
2. Tartaglia L A, et al. Proceedings of the National Academy of Sciences of the United States of America. 1991; 88: 9292-6.
3. Espevik T, et al. Journal of Experimental Medicine. 1990; 171: 415-26.
4. Loetscher H, et al. Journal of Biological Chemistry. 1993; 268: 26350-7.
5. Van Ostade X, et al. European Journal of Biochemistry. 1994; 220: 771-779.
6. Barbara J A, et al. EMBO Journal. 1994; 13: 843-50.
7. Engelmann H, et al. J. Biol. Chem. 1990; 265: 14497.
8. Bigda J, et al. Journal of Experimental Medicine. 1994; 180: 445-60.
9. Tartaglia L A, et al. Journal of Biological Chemistry. 1993; 268: 18542-8.
10. Vandenabeele P, et al. Journal of Experimental Medicine. 1992; 176: 1015-24.
11. Naume B, et al. Journal of Immunology. 1991; 146: 3045-8.
12. Grell M, et al. Cell. 1995; 83: 793-802.
13. Carswell E A, et al. Proc. Natl. Acad. Sci. USA. 1975; 72: 3666-70.
14. Helson L, et al. Nature. 1975; 258: 731-732.
15. Tracey K J and Cerami A. Annual Review of Cell Biology. 1993; 9: 31743.
16. Elliott M J, et al. International Journal of Immunopharmacology. 1995; 17: 141-5.
17. Palladino M A, Jr., et al. Journal of Immunology. 1987; 138: 4023-32.
18. Clauss M, et al. Journal of Biological Chemistry. 1990; 265: 7078-83.
19. Nawroth P P and Stem D M. Journal of Experimental Medicine. 1986; 163: 740-5.
20. Clauss M, et al. Journal of Experimental Medicine. 1990; 172: 1535-45.
21. McIntosh J K, et al. Cancer Research. 1990; 50: 2463-9.
22. Meulders Q, et al. Kidney International. 1992; 42: 327-34.
23. van de Wiel P A, et al. Immunopharmacology. 1992; 23: 49-56.
24. Nawroth P, et al. Journal of Experimental Medicine. 1988; 168: 637-47.
25. Stryhn Hansen A, et al. European Journal of Immunology. 1993; 23: 2358-64.
26. Taylor A. FASEB Journal. 1993; 7: 290-8.
27. Shipp M A and Look A T. Blood. 1993; 82: 1052-70.
28. Fraker D L, Alexander H R and Pass H I: Biologic therapy with TNF: systemic administration and isolation-perfusion. in Biologic therapy of cancer: principles and practice. V. De Vita, S. Hellman and S. Rosenberg, ed. J.B. Lippincott Company:Philadelphia. 1995.329-345.
29. Fiers W: Biologic therapy with TNF: preclinical studies. in Biologic therapy of cancer: principles and practice. V. De Vita, S. Hellman and S. Rosenberg, ed. J.B. Lippincott Company:Philadelphia. 1995.295-327.
30. Sidhu R S and Bollon A P. Pharmacological Therapy. 1993; 57: 79-128.
31. Hieber U and Heim M E. Oncology. 1994; 51: 142-53.
32. Lienard D, et al. World Journal of Surgery. 1992; 16: 234-40.
33. Hill S, et al. British Journal of Surgery. 1993; 80: 995-7.
34. Eggermont A M, et al. Annals of Surgery. 1996; 224: 756-65.
35. Schraffordt Koops H, et al. Radiotherapy and Oncology. 1998; 48: 1-4.
36. Williamson B D, et al. Proceedings of the National Academy of Sciences of the United States of America. 1983; 80: 5397-401.
37. Fransen L, et al. European Journal of Cancer & Clinical Oncology. 1986; 22: 419-26.
38. Ruff M R and Gifford G E: Tumor Necrosis Factor. in Lymphokines. E. Pick, ed. Academic Press:New York. 1981.235-272.
39. Beyaert R, et al. Cancer Research. 1993; 53: 2623-30.
40. Beyaert R, et al. Proceedings of the National Academy of Sciences of the United States of America. 1989; 86: 9494-8.
41. Balkwill F R, et al. Cancer Research. 1986; 46: 3990-3.
42. Schiller J H, et al. Cancer. 1992; 69: 562-71.
43. Jones A L, et al. Cancer Chemotherapy & Pharmacology. 1992; 30: 73-6.
44. Brouckaert P, et al. Lymphokine & Cytokine Research. 1992; 11: 193-6.
45. Van Ostade X, et al. Nature. 1993; 361: 266-9.
46. Van Zee K J, et al. Journal of Experimental Medicine. 1994; 179: 1185-91.
47. Bartholeyns J, et al. Infection & Immunity. 1987; 55: 2230-3.
48. Debs R J, et al. Journal of Immunology. 1989; 143: 1192-7.
49. Debs R J, et al. Cancer Research. 1990; 50: 375-80.
50. Kimura K, et al. Cancer Chemotherapy & Pharmacology. 1987; 20: 223-9.
51. Aderka D, et al. Cancer Research. 1991; 51: 5602-7.
52. Lantz M, et al. Cytokine. 1990; 2: 402-6.
53. Hoogenboom H R, et al. Molecular Immunology. 1991; 28: 1027-37.
54. Yang J, et al. Human Antibodies & Hybridomas. 1995; 6: 129-36.
55. Yang J, et al. Molecular Immunology. 1995; 32: 873-81.
56. Pasqualini R, et al. Nature Biotechnology. 1997; 15: 542-6.
57. Koivunen E, et al. Nature Biotechnology. 1999; 17: 768-774.
58. Brekken R A, et al. Cancer Research. 1998; 58: 1952-1959.
59. Goodwin D A. Journal of Nuclear Medicine. 1995; 36: 876-9.
60. Paganelli G, et al. Cancer Research. 1991; 51: 5960-6.
61. Modorati G, et al. British Journal of Ophtalmology. 1994; 78: 19-23.
62. Colombo P, et al. Journal of Endocrinological Investigation. 1993; 16: 841-3.
63. Paganelli G, Magnani P, Siccardi A and Fazio F: Clinical application of the avidin-biotin system for tumor targeting. in Cancer therapy with radiolabeled antibodies. D. Goldenberg, ed. CRC Press:Boca Raton. 1995.239-253.
64. Robert B, et al. Cancer Research. 1996; 56: 4758-4765.
65. Corti A, et al. Cancer Research. 1998; 58: 3866-3872.
66. Pennica D, et al. Proceedings of the National Academy of Sciences of the United States of America. 1985; 82: 6060-4.
67. Corti A, et al. Journal of Immunological Methods. 1994; 177: 191-198.
68. Moro M, et al. Cancer Research. 1997; 57: 1922-8.
69. Ljunggren H G and Karre K. Journal of Experimental Medicine. 1985; 162: 1745-59.
70. Celik C, et al. Cancer Research. 1983; 43: 3507-10.
71. Gasparri A, et al. Cancer Research. 1999; 59: 2917-23.
72. Arap W, et al. Science. 1998; 279: 377-80.
73. Talmadge J E, et al. Cancer Research. 1987; 47: 2563-70.
74. Pfizemaier K, et al. Journal of Immunology. 1987; 138: 975-80.
75. Asher A L, et al. Journal of Immunology. 1991; 146: 3227-34.
76. Mizuguchi H, et al. Cancer Research. 1998; 58: 5725-30.
77. Gasparri A, et al. Journal of Biological Chemistry. 1997; 272: 20835-43.

1. A conjugate of a cytokine and a tumor targeting moiety (TTM) with the provisos that when the cytokine is TNF-α, TNF-β or IFN-γ, the TTM is other than a CD13 ligand; when the cytokine is IL-2 or IL-12, the TTM is other than an antibody to fibronectin; when the cytokine is TNF, the TTM is other than an antibody to the transferrin receptor; when the cytokine is TNF, IFN-γ or IL-2 the TTM is other than an antibody to the TAG72 antigen; when the cytokine is IFN, the TTM is other than αvβ3 integrin ligand and when the cytokine is TNF, the TTM is other than fibronectin.

2. A conjugate according to claim 1 with the further proviso that when the cytokine is TNF-α or TNF-β, the TTM is other than a tumor specific antibody.

3. A conjugate according to claim 1 with the further proviso that the conjugate is not biotinylated TNF.

4. A conjugate according to claim 1 wherein the cytokine is an inflammatory cytokine.

5. A conjugate according to claim 1 wherein the cytokine is a chemotherapeutic cytokine.

6. A conjugate according to any preceding claim 1 wherein the cytokine is TNFα, TNFβ, IFNα, IFNβ, IFNγ, IL-1, 2, 4, 6, 12, 15, EMAP II, vascular endothelial growth factor (VEGF), PDGF, PD-ECGF or a chemokine.

7. A conjugate according to claim 1 wherein the cytokine is TNF-α, TNF-β or IFN-γ.

8. A conjugate according to claim 1 wherein the TTM is a tumor vasculature targeting moiety (TVTM).

9. A conjugate according to claim 8 wherein the TVTM is a binding partner of a tumor vasculature receptor, marker or other extracellular component, such as a peptide which targets the tumor vasculature.

10. A conjugate according to of claim 1 wherein the TTM is a binding partner of a tumor receptor, marker or other extracellular component.

11. A conjugate according to claim 1 wherein the TTM is an antibody or ligand, or a fragment thereof.

12. A conjugate according to claim 1 wherein the TTM is contains the NGR or RGD motif, or is HIV-tat, Annexin V, Osteopontin, Fibronectin, Collagen Type I or IV, Hyaluronate, Ephrin, or is a binding partner to oncofetal fibronectin; or a fragment thereof.

13. A conjugate according to claim 1 wherein the TTM contains the NGR motif.

14. A conjugate according to claim 13 wherein the TTM is CNGRCVSGCAGRC, NGRAHA, GNGRG, cycloCVLNGRMEC, linear or cyclic CNGRC.

15. A conjugate according to claim 1 wherein the TTM contains the RGD motif.

16. A conjugate according to claims 1 wherein the TTM is targeted to VEGFR, ICAM 1, 2 or 3, PECAM-1, CD31, CD13, VCAM-1, Selectin, Act R11, ActRIIB, ActRI, ActRIB, CD44, aminopeptidase A, aminopeptidase N (CD13), αvβ3 integrin, αvβ5 integrin, FGF-1, 2, 3, or 4, IL-1R, EPHR, MMP, NG2, tenascin, oncofetal fibronectin, PD-ECGFR, TNFR, PDGFR or PSMA.

17. A conjugate according to claim 1 as listed in Table A.

18. A conjugate according to claim 1 wherein the conjugate is in the form of a fusion protein.

19. A conjugate according to claim 1 wherein the conjugate is in the form of nucleic acid.

20. An expression vector comprising the nucleic acid of claim 19.

21. A host cell transformed with the expression vector of claim 20.

22. A method for preparing a conjugate comprising culturing the host cell of claim 21 under conditions which provide for the expression of the conjugate.

23. A pharmaceutical composition comprising the conjugate of claim 1, together with a pharmaceutically acceptable carrier, diluent or excipient.

24. A pharmaceutical composition according to claim 23 wherein the composition further comprises another antitumor agent or diagnostic tumor-imaging compound.

25. A pharmaceutical composition according to claim 24 wherein the further antitumor agent is doxorubicin or melphalan.

26. [canceled]

27. A method of treating or diagnosing cancer comprising administering to a patient in need of the same an effective amount of a conjugate as defined in claim 1.

28. A pharmaceutical composition comprising an effective amount of a conjugation product of TNF and a first TTM or a polynucleotide encoding the same, and an effevctive amount of IFN-γ and a second TTM or a polynucleotide encoding the same, wherein said first TTM and said secoond TTM compete for different receptors.

29. A composition according to claim 27 together with a pharmaeutically acceptable carrier, diluent or excipient.

30. A compostion according to claim 27 wherein said first or said second TTM is a ligand of the CD13 receptor.

31. A composition according to claim 27 wherein said first or said second TTM contains the NGR motif.

32. A composition according to claim 27 wherein said first or said second TTM is CNGRCVSGCAGRC, NGRAHA, GNGRG, cycloCVLNGRMEC, linear or cyclic CNGRC.

33. A composition according to claim 27 wherein said first or said second TTM is a ligand of the αvβ3 receptor.

34. A composition according to claim 27 wherein said first or said second TTM contains the RGD motif.

35. A composition according to claim 27 wherein said first TTM is a ligand of the CD13 receptor and said second TTM is a ligand of the αvβ3 receptor.

36. A composition according to claim 27 wherein said first TTM is a ligand of the αvβ3 receptor and said second TTM is a ligand of the CD13 receptor.

37. A conjugate according to claim 2 with the further proviso that the conjugate is not biotinylated TNF.
